(19) United States
(12) Patent Application Publication
(10) Pub. No.: US 2023/0241256 A1
(43) Pub. Date: Aug. 3, 2023

(54) NON-INTUITIVE COMBINATION OF DRUG DELIVERY CARRIERS OF THE SAME DRUG FOR SYNERGISTIC GROWTH DELAY OF SOLID TUMORS

(71) Applicant: The Johns Hopkins University, Baltimore, MD (US)

(72) Inventors: Stavroula Sofou, Baltimore, MD (US); Alaina Karen Howe, Baltimore, MD (US)

(21) Appl. No.: 18/011,071

(22) PCT Filed: Jun. 30, 2021
(86) PCT No.: PCT/US2021/039885

§ 371 Date: Dec. 16, 2022

Related U.S. Application Data
(60) Provisional application No. 63/046,256, filed on Jun. 30, 2020.

(51) Int. Cl. A61K 51/04; A61K 51/10; C07K 16/28; A61K 9/127; A61P 35/00; C07K 16/30; A61K 39/00

(52) U.S. Cl. CPC A61K 51/0482; A61K 51/1093; A61K 51/1051; C07K 16/2863; A61K 9/1271; A61P 35/00; C07K 16/3069; A61K 2039/505

(57) ABSTRACT

The disclosure is directed to method of inhibiting cancer cell growth by contacting cancer cells with a dose of an anti-cancer agent that is divided equally between a nanoparticle carrier encapsulating the anti-cancer agent and antibody carrier that binds to a cancer-specific receptor and is conjugated to the same anti-cancer agent.

### STATEMENT REGARDING FEDERALLY-SPONSORED RESEARCH

This invention was made with government support under CA058236 awarded by the National Institutes of Health. The government has certain rights in the invention.

### BACKGROUND

Metastatic and/or recurrent solid cancers, such as breast and prostate cancer, are among the leading sites of new cancer cases and deaths in the U.S. (American Cancer Society; Cancer Facts & Figures. 2020). This occurrence is partly due to the development of resistance to existing therapeutics, thereby limiting the available therapeutic options for these patients. Vasan et al., 2019. Therapy with radiopharmaceuticals (pharmaceutical drugs containing radioactive isotopes) has been effective, but is confined to the treatment of disseminated small metastases. Navarro-Teulon et al., 2013. A treatment that targets established (i.e., large, vascularized) lesions in conjunction with the targeting of small metastases is critical to successfully treating solid tumor patients at every stage of their disease. Clinical studies with α-particle emitters have produced some exceptional outcomes in patients with metastatic prostate cancer resistant to approved treatments (Parsad et al., 2020; Kratochwil et al., 2016; Poty et al., 2018). Alpha-particle therapies, however, have not yet shown success against established tumors, partly due to delivery-related issues. McDevitt et al., 2018. Thus, there remains a need for compositions and methods that efficiently deliver radiopharmaceuticals to established cancers to inhibit tumor growth and reduce metastases.

### SUMMARY

In some aspects, the presently disclosed subject matter provides a method for inhibiting cancer cell growth, the method comprising contacting one or more cancer cells with a therapeutically effective amount of a first composition comprising a nanoparticle encapsulating an anti-cancer agent and a second composition comprising an antibody that binds to a cancer-specific receptor and is conjugated to the same anti-cancer agent comprising the first composition, whereby the first and second compositions are delivered to the cancer cells, thereby inhibiting cancer cell growth.

In certain aspects, the anti-cancer agent comprises a radiopharmaceutical agent. In particular aspects, the anti-cancer agent comprises an alpha-particle emitting radiopharmaceutical agent. In more particular aspects, the alpha-particle emitting radiopharmaceutical agent comprises Actinium-225 (²²⁵Ac). In certain aspects, the anti-cancer agent comprises a chemotherapeutic agent.

In certain aspects, the nanoparticle comprises a cationic polymer attached to the surface thereof. In particular aspects, the cationic polymer comprises polyethylene glycol (PEG) conjugated to dimethyl ammonium propane (DAP). In certain aspects, the nanoparticle comprises a pH-responsive membrane capable of forming phase-separated domains upon pH lowering.

In certain aspects, the nanoparticle adheres to extracellular matrix of the one or more cancer cells. In certain aspects, the anti-cancer agent is released from the nanoparticle into the interstitium of the cancer cells.

In certain aspects, the antibody binds to a cancer-specific receptor selected from HER2, epidermal growth factor receptor (EGFR), vascular endothelial growth factor receptor (VEGFR), interleukin-4 (IL-4), αvβ integrin, insulin-like growth factor receptor 1 (IGFR1), insulin-like growth factor receptor 2 (IGFR1), folate receptor, transferrin receptor, estrogen receptor, CXCR4, interleukin-6 (IL-6), transforming growth factor-beta receptor (TGF-βR), prostate specific membrane antigen (PSMA), α6β1 integrin, IGF1, EphA2, tumor necrosis factor-related apoptosis-inducing ligand (TRAIL), platelet derived growth factor receptor (PDGFR), CD20, and fibroblast growth factor receptor (FGFR). In particular aspects, the antibody is trastuzumab, cetuximab, panitumumab, rituximab, or bevacizumab.

In certain aspects, the one or more cancer cells are from a primary cancer or tumor. In particular aspects, the primary cancer or tumor is located in the breast, pancreas, or prostate. In certain aspects, the one or more cancer cells are from a metastatic cancer or tumor.

In certain aspects, the one or more cancer cells are contacted with the anti-cancer agent in vitro. In certain aspects, the one or more cancer cells are contacted with the anti-cancer agent in vivo. In certain aspects, the one or more cancer cells are in a human.

In certain aspects, delivery of the first composition and the second composition to the one or more cancer cells synergistically lowers the therapeutically effective amount of the anti-cancer agent relative to a therapeutically effective amount of the anti-cancer agent administered in either the first composition or the second composition alone.

In certain aspects, the first composition and the second composition are contacted with the one or more cancer cells simultaneously. In certain aspects, the first composition and the second composition are contacted with the one or more cancel cells sequentially.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 is a schematic diagram illustrating an engineered nanoparticle described herein (right panel) compared to a conventional nanoparticle (left panel);

FIG. 2A is a schematic diagram showing the mechanism of triggered release of contents from the membrane forming the NP. Phase-separation and formation of patches in intratumoral acidic environments are accompanied by formation of extensive defects in the bilayer (arrows). Through these defects encapsulated contents leak out fast and extensively.

FIG. 2B includes a graph and images showing that the distribution of ²²⁵Ac-DOTA within the spheroid volume is almost uniform in the deep parts of spheroids (dark plot) when delivered by NPs that are triggered to release the therapeutic agents in the interstitium, compared to when delivered by conventional NPs that are not designed to release their therapeutic contents (light plot). Zhu et al., 2017;

FIG. 3A includes a graph showing that NPs with the cationic moiety on the free ends of PEG-chains do not significantly interact with cells. FIG. 3B includes a graph showing that NPs with the cationic charge directly on their surface exhibit strong cell binding and internalization. Errors correspond to standard deviations of 3 independent measurements/NP preparations of NP incubated with cancer cells. Stras et al., 2020. The right panel of each figure includes schematic diagrams of NP structure showing different locations of the cationic charge;

FIG. 4A is a graph showing that NPs with the adhesion property (filled symbols) exhibit higher tumor uptake, slower tumor clearance, and higher AUCtumor compared to the same size NP without adhesion (unfilled circles) (p-value for the AUC comparison <0.05) (n=5 animals were I.V. administered ¹¹¹In-DTPA-labeled NP). Stras et al, 2020. FIG. 4B is a graph showing that the adhesion property does not change the blood circulation kinetics of the NPs. FIGS. 4C and 4D are graphs showing that NPs with the adhesion property exhibit a shifted uptake and clearance behavior from the liver (FIG. 4C) and the spleen (FIG. 4D);

FIG. 5A is a graph showing that synergy in controlling tumor growth in vivo is observed when the same total dose of the α-particle emitter Actinium-225 (²²⁵Ac) is equally split between the two carrier modalities (tumor responsive nanoparticles (²²⁵Ac-DOTA NP) and radiolabeled antibodies (²²⁵Ac-DOTA-SCN-Ab)) targeting HER2 in Balb/c nu/nu female mice with HER2+BT474 breast cancer orthotopic xenografts (p-value: * <0.01; N=8-10 mice per group). FIG. 5B is an image of H&E stained tumor slice sections demonstrating a significant decrease of cancer cells when animals were treated with ²²⁵Ac delivered by cocktails of ²²⁵Ac-DOTA NP and ²²⁵Ac-DOTA-SCN-Ab relative to any carrier modality alone (scale bar=100 μm). Howe and Sofou, 2020. FIG. 5C is a graph showing that synergy in controlling tumor growth in vivo is observed when the same total dose of Actinium-225 (²²⁵Ac) is equally split between the two carrier modalities (tumor responsive nanoparticles (²²⁵Ac-DOTA NP) and radiolabeled antibodies (²²⁵Ac-DOTA-SCN-Ab) targeting PSMA in NOD scid gamma male mice with PSMA+PC3-PIP prostate cancer xenografts (p** <0.001; N=8-10 mice per group);

FIG. 6A, FIG. 6B, and FIG. 6C are alpha-camera images of tumor slices showing the normalized pixel intensities of ²²⁵Ac relative to the average of all pixels in the entire tumor slice, so as to evaluate the range of heterogeneities in ²²⁵Ac-microdistributions (the scale represents each pixel divided with the average of all pixels in the entire tumor). Tumors treated with the same total dose delivered by only the NP (FIG. 6A) or only the Ab (FIG. 6B) exhibited large regions indicative of too low of a locally delivered dose (minimum intensity ratios=0.238 and 0.033 below the average of the whole tumor). FIG. 6C shows that treatment with both NP+Ab demonstrated more uniform microdistributions (pixel ratio around 1 for most of the tumor);

FIG. 7 shows that microdistributions in spheroids of ²²⁵Ac delivered by tumor-responsive NPs and targeting Abs are complementary. The top panels show alpha-camera images of equatorial slices of spheroids of HER2+ breast cancer cells. The panel labeled (**) shows that treatment with conventional NP (non-releasing, non-adhering) resulted in irradiation limited to spheroid periphery (**); panel (A) shows that the tumor-responsive NP released the highly diffusing ²²⁵Ac-DOTA in the interstitium, resulting in uniform irradiation of the deep parts of spheroids but in limited irradiation of the periphery due to fast clearance of released ²²⁵Ac-DOTA; and panel (B) shows that strong irradiation by targeting ²²⁵Ac-labeled Abs is limited to the spheroid periphery due to the Abs' limited penetration (Binding Site Barrier Effect (Graff and Wittrup, 2003)). Panel C and the bottom panel show schematics of the strategy to leverage the complementary tumor micro-distributions of the two types of carriers (scale bar: 400 m);

FIG. 8A, FIG. 8B, FIG. 8C, FIG. 8D, FIG. 8E, FIG. 8F, and FIG. 8G are graphs illustrating the feasibility of using the disclosed method in different cancers with variable expression of a targeted surface marker. The data demonstrate that HER2-overexpressing BT474 breast cancer spheroids (FIG. 8A), HER1-(moderately) expressing MDA-MB-231 triple negative breast cancer spheroids (FIG. 8B), PSMA positive LNCaP prostate cancer spheroids (FIG. 8C), PSMA positive C42b prostate cancer spheroids (FIG. 8D), HER1 positive triple negative breast cancer spheroids (FIG. 8E), and HER1 positive pancreatic cancer spheroids (FIG. 8F) are each much better controlled with the NP+Ab cocktails (see arrow) than by each carrier modality alone (* indicates p-values <0.01). FIG. 8G is a graph showing that, in spheroids, the NP+Ab cocktail produces the same synergy in controlling spheroid outgrowth that is independent of introducing both carrier modalities concurrently or each modality separately with at least a 72-hour lag period;

FIG. 9A, FIG. 9B, FIG. 9C, and FIG. 9D show colony survival of Trastuzumab-sensitive BT474 (top panel, FIG. 9A and FIG. 9B, 1.50±0.10×10⁶ HER2 copies/cell) and Trastuzumab-resistant BT474-R (lower panel, FIG. 9C and FIG. 9D, 0.93±0.04×10⁶ HER2 copies/cell) breast cancer cells following a 6 hour incubation at 37° C. with free [²²⁵Ac]Ac-DOTA (gray symbols), tumor-responsive liposomes loaded with [²²⁵Ac]Ac-DOTA (black symbols) and radiolabeled Trastuzumab ([²²⁵Ac]Ac-DOTA-SCN-Ab) (white symbols) at extracellular pH values of 7.4 (FIG. 9A and FIG. 9C) or 6.0 (FIG. 9B and FIG. 9D, as the lowest expected acidic value of the tumor interstitial pHₑ). Radiolabeled Trastuzumab's specific activity was 2.9 MBq/mg (78.3 μCi/mg) at the highest radioactivity concentration. Cold conditions of liposomes and the antibody are indicated at zero radioactivity concentration. Error bars correspond to standard deviations of repeated measurements (4-6 samples per radioactivity concentration);

FIG. 10A, FIG. 10B, and FIG. 10C show time-integrated concentrations in HER2-positive BT474 spheroids (r=200 μm) of (FIG. 10A) the fluorescently labeled antibody (AlexaFluor-647-NHS-Trastuzumab) used as surrogate of [²²⁵Ac]Ac-DOTA-SCN-Trastuzumab, (FIG. 10B) the lipids (DPPE-Rhodamine-labeled liposomes), and (FIG. 10C) the CFDA-SE fluorophores (used as surrogates of [²²⁵Ac]Ac-DOTA) delivered by tumor-responsive liposomes. The spatial distributions obtained at different timepoints (during carrier uptake by and clearance from spheroids) were integrated using the trapezoid rule along the spheroid radius. Error bars correspond to the propagated standard deviations of the measurements of n=3-6 equatorial spheroid sections per time point. Immunoreactivity of the fluorescently-labeled antibody was: 88.2±2.7%;

FIG. 10D, FIG. 10E, and FIG. 10F show that the greatest suppression of the extent of outgrowth (used as an indirect surrogate of tumor recurrence) by a carrier, or combinations of carriers, of ²²⁵Ac depends on spheroid size (representing tumor avascular regions). Outgrowth control (FIG. 10D) of small spheroids (radius=100 μm) was best enabled (indicated by arrow) by radiolabeled antibodies ([225Ac]Ac-DOTA-SCN-Trastuzumab), (FIG. 10F) of large spheroids (radius=300 μm) by tumor-responsive liposomes encapsulating [²²⁵Ac]Ac-DOTA, and (FIG. 10E) of medium size spheroids (radius=200 μm) by dividing the same total radioactivity between both carriers. The total radioactivity concentration was kept constant per spheroid size (FIG. 10D) 9.25 kBq/mL, (FIG. 10E) 13.75 kBq/mL, and (FIG. 10F) 18.5 kBq/mL. Error bars correspond to the standard deviations of repeated measurements (n=4-5 spheroids per condition, 2 independent preparations). ** indicates 0.001<p-values<0.01; *** indicates p-values<0.001;

FIG. 11 shows tumor and non-tumor extracellular pH (pHₑ) maps of two different animals with orthotopic BT474 xenografts on NCR nu/nu female mice which were administered I.P. with ISUCA, were imaged by MRSI. pHₑ maps are presented overlaid with MRI anatomical images of the tumors. The ISUCA chemical shift for each voxel (1×1×4 mm³) of the acquired multivoxel spectroscopy grid was transformed into a pH value using the Henderson-Hasselbalch calibration curve and presented as a colored pHₑ map;

FIG. 12A, FIG. 12B, and FIG. 12C show the biodistributions in mice bearing orthotopic BT474 xenografts of I.V. administered (FIG. 12A) [¹¹¹In]In-DTPA-SCN-Trastuzumab and (FIG. 12B) tumor-responsive liposomes encapsulating [¹¹¹In]In-DTPA. (FIG. 12C) Comparison of blood clearance and tumor uptake/clearance of each carrier. Error bars correspond to standard deviations of measurements averaged over n=3 mice per time point. Radiolabeling stability of Trastuzumab with ¹¹¹In (89.3±4.4%, 24-hour radioactivity retention) and retention of [¹¹¹In]In-DTPA by liposomes was similar to Trastuzumab radiolabeling with ²²⁵Ac and to retention of ²²⁵Ac-DOTA by liposomes, respectively (Table 1, Prasad et al., 2021);

FIG. 13A, FIG. 13B, and FIG. 13C show the microdistributions of the same total radioactivity of ²²⁵Ac delivered by (FIG. 13A) tumor-responsive liposomes only, (FIG. 13B) the radiolabeled Trastuzumab only, and (FIG. C) by both liposomes and the, separately administered, Trastuzumab, on tumor sections harvested 24 hours post I.V. administration of 4 μCi per animal. High radioactivity relative levels (ratios>2, purple) were detected in densely vascularized tumor areas (CD31+, indicated in green inserts); low radioactivity relative levels (ratios<0.6) were detected in sparsely vascularized areas (yellow inserts). Top panel: Map of normalized pixel intensities (ratios) of ²²⁵Ac relative to the mean value of intensities averaged over the entire tumor section, so as to evaluate the range of heterogeneities in ²²⁵Ac-microdistributions. Regions in red (with ratios around unity) indicate local distributions close to the mean tumor-delivered radioactivities. Regions in cyan and dark-blue (with normalized pixel intensity ratios well-below the mean tumor-delivered radioactivities) indicate regions with low or too low radioactivities relative to the tumor mean, expected to result in less cell kill. Middle and Bottom panels: Decay-corrected α-Camera images, and H&E-, and CD31-stained images of sequential 16 μm-thick-tumor sections;

FIG. 14A shows volume progression of HER2-positive BT474 orthotopic xenografts on NCR nu/nu female mice following a single I.V. administration (indicated by the black arrow) of 9.25 kBq (250 nCi) per 20 g mouse of ²²⁵Ac delivered by the radiolabeled Trastuzumab alone ([225Ac]Ac-DOTA-SCN-antibody, 2.96 MBq/mg specific radioactivity in injectate) (white circles), the tumor-responsive liposomes loaded with [²²⁵Ac]Ac-DOTA alone (black circles), by both carriers at equally split (same total) radioactivity with the radiolabeled antibody being administered 72 hours after the liposomes (to largely allow for the clearance of the latter from the liver and spleen) (half-black-half-gray circles), and by both carriers at equally split (same total) radioactivity injected simultaneously (half-black-half-white circles). Data points are the mean values and error bars the standard deviations of n=8-9 animals per group. Significance was calculated with one-way ANOVA (p-value<0.05). * indicates 0.01<p-values<0.05; **0.001<p-values<0.01; and FIG. 14B show H&E stained tumor sections. Scale bar=100 μm.

FIG. 15 shows tumor growth inhibition, survival and/or elimination of spontaneous metastases is maximized when a single injection of the same total radioactivity per animal (80 nCi/20 g mouse) is split between two separate carriers (nanoparticles, NP, and antibodies, Ab,—not connected to each other) due to more uniform tumor micro-distributions of delivered α-particles, see FIG. 9. Receptor expression ≥1+ is adequate for the presently disclosed approach to deliver lethal doses in the tumor perivascular regions by any FDA approved antibody targeting the particular cell surface marker; and

FIG. 16 shows the effect of the dose split ratio: Radioactivities equally split (50:50) between the two carriers (nanoparticles, NP, and antibodies, Ab,—not connected to each other) resulted in best tumor growth inhibition after a single injection of the same total radioactivity per animal (125 nCi/20 g mouse). Both carriers were injected at the same time. Animal model: PSMA-positive (3+ by IHC score) PC3-PIP prostate cancer subcutaneous xenografts on NOD SCID male mice. A PSMA-targeting antibody was used (that was a gift from Progenics (see Zhu et al., 2016)).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The present disclosure is predicated, at least in part, on the development of a novel, transport-driven delivery strategy using next-generation nanoparticles in combination with established cancer-targeting antibodies to effectively deliver α-particles to established tumors. As described below, this strategy has been found to inhibit tumor growth and delay the spreading of new metastases, independent of resistance to other agents and disease stage. The disclosed method delivers a large number of α-particles at the periphery of a tumor where the cell number is greatest and where cells are growing most aggressively, and simultaneously delivers a high capacity penetrating payload to the tumor interior, where dormant and resistant cells are most likely to be responsible for treatment failure. The disclosed method overcomes existing delivery obstacles while simultaneously reducing potential toxicity due to the lower doses needed to inhibit tumor growth.

In particular, the disclosed method involves a novel nanotechnology platform in which nanoparticles (NPs) have been engineered to (a) trigger release of encapsulated radionuclide contents in the tumor interstitium, and (b) trigger adhesion of NPs primarily to the tumor extracellular matrix (ECM) with minimal internalization by cells. This nanotechnology platform has been designed to carry α-particle emitters that potentiate uniform irradiation of the deep parts of a tumor and maximize retention of the emitted energy. A novel transport-driven strategy also is employed which combines the nanotechnology platform with established targeting modalities based on the complementarity of their individual tumor micro-distributions.

### Definitions

To facilitate an understanding of the present technology, a number of terms and phrases are defined below. Additional definitions are set forth throughout the detailed description.

The term “tumor,” as used herein, refers to an abnormal mass of tissue that results when cells divide more than they should or do not die when they should. In the context of the present disclosure, the term tumor may refer to tumor cells and tumor-associated stromal cells. Tumors may be benign and non-cancerous if they do not invade nearby tissue or spread to other parts of the organism. In contrast, the terms “malignant tumor,” “cancer,” and “cancer cells” may be used interchangeably herein to refer to a tumor comprising cells that divide uncontrollably and can invade nearby tissues. Cancer cells also can spread or “metastasize” to other parts of the body through the blood and lymph systems. The terms “primary tumor” or “primary cancer” refer to an original, or first, tumor in the body. The term “metastasis,” as used herein, refers to the process by which cancer spreads from the location at which it first arose as a primary tumor to distant locations in the body. The terms “metastatic cancer” and “metastatic tumor” refer to the cancer or tumor resulting from the spread of a primary tumor. It will be appreciated that cancer cells of a primary tumor can metastasize through the blood or lymph systems.

An agent is “cytotoxic” and induces “cytotoxicity” if the agent kills or inhibits the growth of cells, particularly cancer cells. In some embodiments, for example, cytotoxicity includes preventing cancer cell division and growth, as well as reducing the size of a tumor or cancer. Cytotoxicity of tumor cells may be measured using any suitable cell viability assay known in the art, such as, for example, assays which measure cell lysis, cell membrane leakage, and apoptosis. For example, methods including but not limited to trypan blue assays, propidium iodide assays, lactate dehydrogenase (LDH) assays, tetrazolium reduction assays, resazurin reduction assays, protease marker assays, 5-bromo-2′-deoxy-uridine (BrdU) assays, and ATP detection may be used. Cell viability assay systems that are commercially available also may be used and include, for example, CELLTITER-GLO® 2.0 (Promega, Madison, Wis.), VIVAFIX™ 583/603 Cell Viability Assay (Bio-Rad, Hercules, Calif.); and CYTOTOX-FLUOR™ Cytotoxicity Assay (Promega, Madison, Wis.).

The term “immunoglobulin” or “antibody,” as used herein, refers to a protein that is found in blood or other bodily fluids of vertebrates, which is used by the immune system to identify and neutralize foreign objects, such as bacteria and viruses. Typically, an immunoglobulin or antibody is a protein that comprises at least one complementarity determining region (CDR). The CDRs form the “hypervariable region” of an antibody, which is responsible for antigen binding. A whole antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H1}, C_{H2}, and C_{H3}) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. The V_{H} and V_{L} regions have the same general structure, with each region comprising four framework (FW or FR) regions. The term “framework region,” as used herein, refers to the relatively conserved amino acid sequences within the variable region which are located between the CDRs. In a typical antibody, each light chain is linked to a heavy chain by disulphide bonds, and the two heavy chains are linked to each other by disulphide bonds. The light chain variable region is aligned with the variable region of the heavy chain, and the light chain constant region is aligned with the first constant region of the heavy chain. The remaining constant regions of the heavy chains are aligned with each other. The variable regions of each pair of light and heavy chains form the antigen binding site of an antibody. (see, e.g., C. A. Janeway et al. (eds.), Immunobiology, 5th Ed., Garland Publishing, New York, N.Y. (2001)).

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23(9): 1126-1129 (2005)). An antibody fragment can comprise, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and C_{H1} domains, (ii) a F(ab′)2 fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)2 fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

“Binding” as used herein (e.g., with reference to a nanoparticle and/or antibody binding to cancer cells) refers to a non-covalent interaction between macromolecules (e.g., between a protein and a nucleic acid or a protein and a protein). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻⁴ M, or less than 10⁻¹⁵ M. “Affinity” refers to the strength of binding, increased binding affinity being correlated with a lower K_{d}. With respect to antibodies in particular, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus.

The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones.

The term “antigen,” as used herein, refers to any subunit, fragment, or epitope of any proteinaceous or non-proteinaceous (e.g., carbohydrate or lipid) molecule that provokes an immune response in a mammal. By “epitope” is meant a sequence of an antigen that is recognized by an antibody or an antigen receptor. Epitopes also are referred to in the art as “antigenic determinants.” In some embodiments, an epitope is a region of an antigen that is specifically bound by an antibody. In other embodiments, an epitope may include chemically active surface groupings of molecules such as amino acids, sugar side chains, phosphoryl, or sulfonyl groups. An epitope may have specific three-dimensional structural characteristics (e.g., a “conformational” epitope) and/or specific charge characteristics.

As used herein, the term “preventing” refers to prophylactic steps taken to reduce the likelihood of a subject (e.g., an at-risk subject) from contracting or suffering from a particular disease, disorder, or condition. The likelihood of the disease, disorder, or condition occurring in the subject need not be reduced to zero for the preventing to occur; rather, if the steps reduce the risk of a disease, disorder or condition across a population, then the steps prevent the disease, disorder, or condition within the scope and meaning herein.

As used herein, the terms “treatment,” “treating,” and the like, refer to obtaining a desired pharmacologic and/or physiologic effect against a particular disease, disorder, or condition. Preferably, the effect is therapeutic, i.e., the effect partially or completely cures the disease and/or adverse symptom attributable to the disease.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

### Anti-Cancer Agents

The disclosure provides a method of inhibiting cancer cell growth which comprises contacting cancer cells with a dose of an anti-cancer agent.

The terms “anti-cancer agent,” “anti-cancer drug,” “anti-cancer therapy,” and “anti-cancer therapeutic,” may be used interchangeably herein to refer to any compound, molecule, substance, or procedure that partially or completely inhibits any or all aspects of cancer development and/or metastases. For example, an anti-cancer agent may inhibit the initiation, promotion, progression, metastasis, and/or neovascularization of a malignant tumor or cancer, as well as any adverse symptoms attributable to the particular cancer. Examples of anti-cancer agents include, but are not limited to, radiation therapeutic agents (e.g., radiopharmaceuticals), chemotherapeutic agents (e.g., alkylating agents, antimetabolites, plant alkaloids, antitumor antibiotics), immunotherapeutic agents (e.g., immune checkpoint inhibitors, monoclonal antibodies, CAR-T cells, cancer vaccines), targeted agents (e.g., small molecule drugs, monoclonal antibodies), and hormone therapies. In some embodiments, the anti-cancer agent is a chemotherapeutic agent, such as, for example, adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, rituximab, streptozocin, teniposide, thioguanine, thiotepa, vinblastine, vincristine, vinorelbine, taxol, transplatinum, anti-vascular endothelial growth factor compounds (“anti-VEGFs”), anti-epidermal growth factor receptor compounds (“anti-EGFRs”), 5-fluorouracil, etc. However, any suitable anti-cancer agent may be used in the context of the present disclosure.

In some embodiments, the anti-cancer agent is a radiopharmaceutical. Radiopharmaceutical therapy (also referred to as “RPT”) involves the use of radionuclides that are either conjugated to tumor-targeting agents (e.g., nanoscale constructs, antibodies, peptides, and small molecules) or that concentrate in tumors through natural physiological mechanisms that occur predominantly in neoplastic cells. The terms “radionuclide,” “radioisotope,” and “radioactive isotope” may be used interchangeably herein to refer to an atom that emits radiation as it undergoes radioactive decay through the emission of alpha particles (a), beta particles (0), or gamma rays (7). RPT agents may be systemically or locally administered for targeting to a tumor or its microenvironment. Tumor targeting may occur because the radioactive element is involved in relevant tumor-associated biological processes or because the radionuclide is conjugated to a delivery vehicle that confers tumor targeting (Sgouros, G., Health Phys., 116(2): 175-178 (2019)). Delivery vehicles that may be used for RPT include, but are not limited to, microspheres, nanoparticles, antibodies, peptides, and small molecules. Several RPT agents have been approved by the U.S. Food and Drug Administration (FDA) or are currently under investigation. Radioiodine (¹³¹I) is a well-known treatment for metastatic cancer, particularly thyroid cancer.

Alpha-particle radiopharmaceutical therapy (αRPT) has shown promise in difficult-to-treat cancers, such as metastatic castration-resistant prostate cancer and triple-negative breast cancer (Kratochwil et al., 2016; Song et al., 2013). The highly efficient irradiation of α-particle emitters (1-10 MeV energy), endows α-particles with a 3- to 8-fold greater relative biological effectiveness compared to photon or β-particle radiation. McDevitt et al., 2018. Alpha particles typically cause double-strand DNA breaks, and their high killing efficacy (1-3 tracks across the nucleus result in cell death) Fournier et al., 2012; Humm et al., 1987; Humm et al., 1993; Macklis et al., 1988, is mostly independent of the cell-oxygenation state and cell-cycle (McDevitt et al., 2018; Sofou, 2008). Hence, the complexity and level of DNA damage induced by αRPT rapidly overwhelms cellular repair mechanisms, and, if optimally delivered, αRPT is impervious to resistance irrespective of cell type or of resistance to other agents (McDevitt et al., 2018; Sgouros, 2019; Yard et al., 2019).

The growing interest in α-particle emitters for cancer therapy is evidenced by the recent FDA approval of [²²³Ra]RaCl₂ (XOFIGO™, Bayer), which targets bone metastases, and the increasing list of clinical trials employing α-particle emitters. Such trials primarily target soft-tissue metastases, and some employ targeted α-particle therapies of Actinium-225 (²²⁵Ac), a powerful α-particle emitter. Indeed, Actinium-225 (²²⁵Ac) is one of the most potent emitters, having a 10-day half-life and yielding 4 α-particles per physical decay to a stable element. Comparatively, the half-life of Francium-221, ²²¹Fr is about 4.9 minutes, the half-life of Astatine-217, ²¹⁷At is about 32 msec, and the half-life of Bismuth-213, ²¹³Bi is about 45.6 min.

The short range of α-particles in tissue (5-10 cell diameters) makes them ideal for precise cell irradiation, but presents challenges for using αRPT to treat established solid tumors. In addition, the diffusion-limited penetration depths of traditional targeted radionuclide vectors (e.g., antibodies) combined with the short range of α-particles result in only partial irradiation of solid tumors, compromising efficacy. That is, tumor regions not hit by the delivered α-particles likely are not killed.

As described further herein, the disclosed methods address the challenges associated with treating established or solid tumors with αRPT by delivering a dose of αRPT using two different carrier modalities with complementary tumor micro-distributions. The combination of carriers collectively enable uniform and prolonged exposure of the entire tumor to effect potent and durable cancer cell kill that is largely impervious to drug resistance.

### Nanoparticles

The inventive method comprises contacting cancer cells with a dose of an anti-cancer agent that is divided between two compositions, wherein the first composition comprises a nanoparticle encapsulating the anti-cancer agent. The term “nanoparticle,” as used herein, refers to a microscopic particle with at least one dimension less than 100 nm. Nanoparticles can be engineered with distinctive compositions, sizes, shapes, and surface chemistries for use in a wide range of biological applications. Such applications include, but are not limited to, drug and gene delivery, fluorescent labeling, probing of DNA structure, tissue engineering, analyte detection, and purification of biomolecules and cells (see, e.g., Salata, O. V., Journal of Nanobiotechnology, volume 2, Article number: 3 (2004); and Wang, E. C. and Wang, A. Z., Integr Biol (Camb)., 6(1): 9-26 (2014)). Any suitable type of nanoparticle may be used in the context of the present disclosure. Exemplary types of nanoparticles (NPs) include liposomes, albumin-bound nanoparticles, polymeric nanoparticles, iron oxide nanoparticles, quantum dots, and gold nanoparticles (Wang and Wang, supra).

In some embodiments, the present disclosure provides nanoparticles (NPs) that are designed to trigger release of a radionuclide encapsulated therein into the tumor interstitium, and to trigger adhesion of the nanoparticles to the extracellular matrix (ECM) of a cancer or tumor with minimal internalization by the tumor or cancer cells. Tumor “interstitium” (also referred to as “interstitial space” and “interstitial fluid”) is situated between the blood and lymph vessels and the tumor or cancer cells, and consists of a solid or matrix phase and a fluid phase, together constituting the tissue microenvironment. The interstitium can be divided into two compartments: the interstitial fluid and the structural molecules of the interstitial or the extracellular matrix (ECM) (Wiig et al., Fibrogenesis Tissue Repair, 3: 12 (2010)).

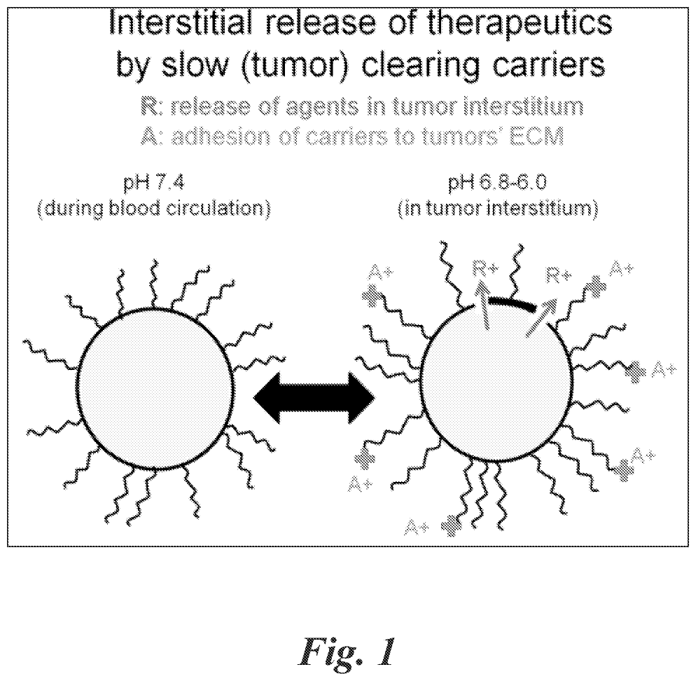

For the property of drug release from NP in the tumor interstitium, nanoparticles may be designed to contain pH-responsive membranes, which can form phase-separated domains (resembling patches) with lowering pH. Bandekar et al., 2012; Karve et al., 2009; Karve et al., 2008; Karve et al., 2010. Thus, in the acidic tumor interstitium, membrane-phase separation may result in the formation of “registered” patches that span the NP bilayer membrane (shown schematically in FIG. 1). Bandekar and Sofou, 2012. This membrane rearrangement may be utilized to create pronounced grain boundaries around the patches, enabling release of the encapsulated therapeutic agents which then may diffuse deeper into solid tumors (Zhu et al., 2017; Stras et al., 2016).

In some embodiments, the property of nanoparticle adhesion to tumor ECM is achieved by generating a positive charge on the outer corona of the nanoparticle. To this end, for example, the nanoparticle may comprise a cationic polymer attached to the surface thereof. In some embodiments, the cationic polymer is attached to an NP and mediates adhesion of the NP to the ECM in the slightly acidic pH of the tumor interstitium (pHₑ-6.7-6.5) (Helmlinger et al., 1997; Vaupel et al., 1989). Any suitable cationic polymer may be used in the context of the present disclosure, including, for example, poly(ethylene glycol) (PEG), gelatin, chitosan, cellulose, dextran, poly(2-N,N-dimethylaminoethylmethacrylate) (PDMAEMA), poly-l-lysine (PLL), and poly(ethyleneimine) (PEI), poly(amidoamine) (PAMAM). Biocompatible cationic polymers are further described in, e.g., Tanaka et al., Polymer Journal, 47: 114-121 (2015); and Farshbaf et al., Artificial Cells, Nanomedicine, and Biotechnology, 46(8): 1872-1891 (2018), doi: 10.1080/21691401.2017.1395344). In some embodiments, the adhesion polymer may comprise poly(ethylene glycol) (PEG) conjugated to the moiety dimethyl ammonium propane (DAP).

The nanoparticles may be produced using any suitable method known in the art, such as those described in, e.g., Naito et al. (eds.), Nanoparticle Technology Handbook, 3rd Edition, Elsevier (2018); Aliofkhazraeil, M. (ed.), Handbook of Nanoparticles, Springer International Publishing, Switzerland (2015); and de la Fuente, J. M. and Grazu, V. (eds.), Nanobiotechnology: Inorganic Nanoparticles vs Organic Nanoparticles, Volume 4, 1st Edition, Elsevier (2012).

### Antibodies

The second of two compositions into which the dose of the anti-cancer agent is divided comprises an antibody that binds to a cancer-specific receptor and is conjugated to the anti-cancer agent. The terms “cancer-specific receptor,” “tumor-specific receptor,” “cancer-specific antigen,” and “tumor-specific antigen,” may be used interchangeably herein to refer to a cell surface receptor that is uniquely expressed by and/or displayed on cancer cells and is not expressed by or displayed on other cells in the body (e.g., normal healthy cells). In contrast, the terms “cancer-associated-receptor,” “tumor-associated-receptor,” “cancer-associated-antigen,” and “tumor-associated-antigen” may be used interchangeably herein to refer to a cell surface receptor that is not uniquely expressed by or displayed on a tumor cell and instead is also expressed on normal cells under certain conditions.

In some embodiments, the antibody is a monoclonal antibody. The term “monoclonal antibody,” as used herein, refers to an antibody produced by a single clone of B lymphocytes that is directed against a single epitope on an antigen. Monoclonal antibodies typically are produced using hybridoma technology, as first described in Kohler and Milstein, Eur. J. Immunol., 5: 511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352: 624-628 (1991)); and Marks et al., J. Mol. Biol., 222: 581-597 (1991)), or produced from transgenic mice carrying a fully human immunoglobulin system (see, e.g., Lonberg, Nat. Biotechnol., 23(9): 1117-25 (2005), and Lonberg, Handb. Exp. Pharmacol., 181: 69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

Monoclonal antibodies that bind to cancer-specific receptors (referred to herein as “cancer-specific” or “tumor-specific” antibodies) typically cause selective cellular toxicity first by binding to a specific target antigen followed by cell lysis via antibody-dependent cellular cytotoxicity, complement activation, complement-dependent cytotoxicity, or by inhibition of signal transduction (e.g. the inhibition of dimerization of a receptor by receptor blocking through a monoclonal antibody) (Attarwala, H., J Nat Sci Biol Med., 1(1): 53-56 (2010)). In the context of the disclosed methods, however, the antibody in the second composition serves primarily to deliver the anti-cancer agent to target cancer cells, and not for any therapeutic effect of the antibody itself. The antibody may bind to any cancer-specific receptor known in the art, as well as cancer-specific receptors not yet identified. Exemplary cancer-specific receptors include, but are not limited to, HER2, epidermal growth factor receptor (EGFR), vascular endothelial growth factor receptor (VEGFR), interleukin-4 (IL-4), αvβ integrin, insulin-like growth factor receptor 1 (IGFR1), insulin-like growth factor receptor 2 (IGFR1), folate receptor, transferrin receptor, estrogen receptor, CXCR4, interleukin-6 (IL-6), transforming growth factor-beta receptor (TGF-DR), prostate specific membrane antigen (PSMA), α6β1 integrin, IGF1, EphA2, tumor necrosis factor-related apoptosis-inducing ligand (TRAIL), platelet derived growth factor receptor (PDGFR), CD20, and fibroblast growth factor receptor (FGFR). Other cancer-specific receptors are described in, e.g., Zeromski J., Arch Immunol Ther Exp (Warsz), 50(2): 105-110 (2002); and Boonstra et al., Biomarkers in Cancer, 8: 119-133 (2016); doi:10.4137/BIC.S38542.

A number of monoclonal antibodies that bind to cancer-specific receptors have been approved to treat a variety of different cancers, any of which may be included in the second composition. Such monoclonal antibodies include, but are not limited to, trastuzumab (HERCEPTIN®, Genentech, Inc.), cetuximab (ERBITUX®, Eli Lilly and Company), panitumumab (VECTIBIX®, Amgen, Inc.), rituximab (RITUXAN®, Genentech, Inc.), and bevacizumab (AVASTIN®, Genentech, Inc.). The disclosure is not limited to these particular antibodies, however, and any antibody that binds to a cancer-specific receptor may be included in the second composition.

In order to deliver the anti-cancer agent to target cancer cells, the antibody desirably is conjugated to the anti-cancer agent. In some embodiments, the antibody is conjugated to the anti-cancer agent using a linker. A linker is any chemical moiety that is capable of linking a compound, usually a drug, to a cell-binding agent such as an antibody or fragment thereof in a stable, covalent manner. Linkers can be susceptible to or be substantially resistant to acid-induced cleavage, light-induced cleavage, peptidase-induced cleavage, esterase-induced cleavage, and disulfide bond cleavage, at conditions under which the antibody remains active. Suitable linkers are well known in the art and include, for example, disulfide groups, thioether groups, acid labile groups, photolabile groups, peptidase labile groups, and esterase labile groups. Linkers also include charged linkers, and hydrophilic forms thereof as described herein and known in the art. In some embodiments, the linker may be a cleavable linker, a non-cleavable linker, a hydrophilic linker, and a dicarboxylic acid based linker. Exemplary linkers that may be used in the disclosed method include, but are not limited to N-succinimidyl 4-(2pyridyldithiojpentanoate (SPP); N-succinimidyl 4-(2-pyridyldithio)-2-sulfopentanoate (sulfoSPP); N-succinimidyl 4-(2-pyridyldithio)butanoate (SPDB); N-succinimidyl 4-(2-pyridyldithio)2-sulfobutanoate (sulfo-SPDB); N-succinimidyl 4-(maleimidomethyl) cyclohexanecarboxylate (SMCC); N-sulfosuccinimidyl 4-(maleimidomethyl) cyclohexanecarboxylate (sulfoSMCC); N-succinimidyl-4-(iodoacetyl)-aminobenzoate (SIAB); and N-succinimidyl-[(Nmaleimidopropionamidoj-tetraethyleneglycol] ester (NHS-PEG4-maleimide).

### Compositions

The nanoparticle and antibody described herein are each separately formulated in a first and second composition, respectively, that each comprises a pharmaceutically acceptable (e.g., physiologically acceptable) carrier. Accordingly, a variety of suitable formulations of the first and second compositions are possible. Methods for preparing compositions for pharmaceutical use are known to those skilled in the art and are described in more detail in, for example, Remington: The Science and Practice of Pharmacy, Lippincott Williams & Wilkins; 21st ed. (May 1, 2005). The choice of carrier will be determined, in part, by the particular use of the compositions (e.g., administration to an animal) and the particular method used to administer the compositions. In some embodiments, the pharmaceutical compositions are sterile.

Suitable compositions include aqueous and non-aqueous isotonic sterile solutions, which can contain anti-oxidants, buffers, and bacteriostats, and aqueous and non-aqueous sterile suspensions that can include suspending agents, solubilizers, thickening agents, stabilizers, and preservatives. The compositions can be presented in unit-dose or multi-dose sealed containers, such as ampules and vials, and can be stored in a freeze-dried (lyophilized) condition requiring only the addition of the sterile liquid carrier, for example, water, immediately prior to use. Each of the nanoparticle and antibody desirably is part of a composition formulated to protect the nanoparticle, antibody, and anti-cancer agent from damage prior to administration to cells. For example, the composition can be formulated to decrease the light sensitivity and/or temperature sensitivity of the nanoparticle, antibody, and/or anti-cancer agent.

One of ordinary skill in the art will appreciate that the nanoparticle or antibody can be present in a composition with other therapeutic or biologically-active agents. For example, factors that control inflammation, such as ibuprofen or steroids, can be part of the composition to reduce swelling and inflammation associated with in vivo administration of the first and/or second composition.

### Inhibiting Cancer Cell Growth

The disclosure provides a method of inhibiting cancer cell growth, which comprises contacting cancer cells with the above-described first and second compositions. Ideally, administration of the first and second compositions described herein inhibits the growth of cancer cells from a primary tumor or primary cancer, such as an established solid tumor. In some embodiments, the method induces cytotoxicity in tumor cells or cancer cells.

A primary cancer or tumor may arise in any organ or tissue. For example, the primary cancer or tumor may be a carcinoma (cancer arising from epithelial cells), a sarcoma (cancer arising from bone and soft tissues), a lymphoma (cancer arising from lymphocytes), a melanoma, or brain and spinal cord tumors. The primary tumor or cancer cells can arise in the oral cavity (e.g., the tongue and tissues of the mouth) and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid). More particularly, primary tumors or cancers of the digestive system can arise in the esophagus, stomach, small intestine, colon, rectum, anus, liver, gall bladder, and pancreas. Primary cancers or tumors of the respiratory system can arise in the larynx, lung, and bronchus and include, for example, non-small cell lung carcinoma. Primary cancers or tumors of the reproductive system can affect the uterine cervix, uterine corpus, ovaries, vulva, vagina, prostate, testis, and penis. Primary cancers of the urinary system can arise in the urinary bladder, kidney, renal pelvis, and ureter. Primary cancer cells also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, etc.). In some embodiments, the cancer cells are from a primary cancer or tumor located in the breast, pancreas, or prostate.

The inventive method comprises contacting cancer cells with a dose of an anti-cancer agent that is equally divided between the first and second compositions described above. The cancer cells may be contacted with the first and second compositions in vitro or in vivo. The term “in vivo” refers to a method that is conducted within living organisms in their normal, intact state, while an “in vitro” method is conducted using components of an organism that have been isolated from its usual biological context. When cancer cells are contacted with the first composition and second composition in vitro, the cell may be any suitable prokaryotic or eukaryotic cell. When cancer cells are contacted with the first composition and second composition in vivo, the compositions may be administered to an animal, such as a mammal, particularly a human, using standard administration techniques and routes. Suitable administration routes include, but are not limited to, oral, intravenous, intraperitoneal, subcutaneous, subcutaneous, or intramuscular administration. The compositions ideally are suitable for parenteral administration. The term “parenteral,” as used herein, includes intravenous, intramuscular, subcutaneous, rectal, vaginal, and intraperitoneal administration. In other embodiments, the compositions may be administered to a mammal using systemic delivery by intravenous, intramuscular, intraperitoneal, or subcutaneous injection.

In some embodiments, the disclosed method promotes inhibition of cancer cell proliferation, the eradication of cancer cells, and/or a reduction in the size of at least one cancer or tumor such that the cancer or tumor is treated in a mammal (e.g., a human). By “treatment of cancer” is meant alleviation of a cancer in whole or in part. In one embodiment, the disclosed method reduces the size of a cancer or tumor by at least about 20% (e.g., at least about 25%, about 30%, about 35%, about 40%, about 45%, about 50%, about 55%, about 60%, about 65%, about 70%, about 75%, about 80%, about 85%, about 90%, or about 95%). Ideally, the cancer or tumor is completely eliminated.

For in vivo applications, any suitable dose of the anti-cancer agent, divided (equally or unequally) before the first composition and the second composition, may be administered to a mammal (e.g., a human), so long as the anti-cancer agent is efficiently delivered to target cancer cells such that cancer cell growth is inhibited. To this end, the inventive method comprises administering a “therapeutically effective amount” of the anti-cancer agent. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the anti-cancer agent to elicit a desired response in the individual. For example, a therapeutically effective amount of the anti-cancer agent is an amount which is cytotoxic to cancer cells, such that the cancer or tumor is eliminated.

Alternatively, the pharmacologic and/or physiologic effect may be prophylactic, i.e., the effect completely or partially prevents cancer cell growth. In this respect, the inventive method comprises administering a “prophylactically effective amount” of the anti-cancer agent. A “prophylactically effective amount” refers to an amount effective, at dosages and for periods of time necessary, to achieve a desired prophylactic result (e.g., prevention of cancer or metastases).

When the anti-cancer agent is an alpha-particle emitting radiopharmaceutical, a typical dose can be, for example, in the range of 50 to 200 kilobecquerel (kBq) per mouse kilogram or per human kilogram; however, doses below or above this exemplary range are within the scope of the invention. The daily dose can be about 50 kBq/kg, about 55 kBq/kg, about 60 kBq/kg, about 65 kBq/kg, about 70 kBq/kg, about 75 kBq/kg, about 80 kBq/kg, about 85 kBq/kg, about 90 kBq/kg, about 95 kBq/kg, about 100 kBq/kg, about 105 kBq/kg, about 110 kBq/kg, about 115 kBq/kg, about 120 kBq/kg, about 125 kBq/kg, about 130 kBq/kg, about 135 kBq/kg, about 140 kBq/kg, about 145 kBq/kg, about 150 kBq/kg, about 155 kBq/kg, about 160 kBq/kg, about 165 kBq/kg, about 170 kBq/kg, about 175 kBq/kg, about 180 kBq/kg, about 185 about kBq/kg, about 190 kBq/kg, about 195 kBq/kg, or a range defined by any two of the foregoing values. Therapeutic or prophylactic efficacy can be monitored by periodic assessment of treated patients. For repeated administrations over several days or longer, depending on the condition, the treatment can be repeated until a desired suppression of disease symptoms occurs. However, other dosage regimens may be useful and are within the scope of the invention.

The disclosed method can be performed in combination with other therapeutic methods to achieve a desired biological effect in a human patient. Ideally, the disclosed method may include, or be performed in conjunction with, one or more cancer treatments. Suitable cancer treatments that may be employed include, but are not limited, surgery, chemotherapy, radiation therapy, immunotherapy, and hormone therapy.

In some embodiments, the administration of a combination of the first composition and the second composition has a synergistic effect. The term “combination” is used in its broadest sense and means that a subject is administered at least two agents, more particularly a first composition and a second composition and, in some embodiments, at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the first composition and the second composition described herein can be administered alone or in combination with adjuvants that enhance stability of the compositions, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the first composition and the second composition and, in some embodiments, at least one additional therapeutic agent, can be varied so long as the beneficial effects of the combination of these agents are achieved. Accordingly, the phrase “in combination with” refers to the administration of a first composition and a second composition, and, in some embodiments, at least one additional therapeutic agent either simultaneously, sequentially, or a combination thereof. Therefore, a subject administered a combination of a first composition and a second composition and, in some embodiments, at least one additional therapeutic agent, can receive a first composition and a second composition and, in some embodiments, at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the first composition and the second composition and, in some embodiments, at least one additional therapeutic agent are administered simultaneously, they can be administered to the subject as separate pharmaceutical compositions, each comprising either a first composition or a second composition or, in some embodiments, at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a first composition and a second compositions, and, in some embodiments, at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index(SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, either the first composition or the second composition or, in some embodiments, another therapeutic agent present in the composition.

### Kits

The first composition and second composition can be provided in a kit, i.e., a packaged combination of reagents in predetermined amounts with instructions for using the compositions (e.g., for administration to a human subject). As such, the disclosure provides a kit comprising the first composition and second composition described herein and instructions for use thereof. The instructions can be in paper form or computer-readable form, such as a disk, CD, DVD, etc. Ideally, the kit comprises all components, i.e., reagents, standards, buffers, diluents, etc., which are necessary to deliver the composition to cells in vitro or in vivo. The kit components may be provided as dry powders (typically lyophilized), including excipients which on dissolution will provide a reagent solution having the appropriate concentration.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments ±50%, in some embodiments ±20%, in some embodiments ±10%, in some embodiments ±5%, in some embodiments ±1%, in some embodiments ±0.5%, and in some embodiments 0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Production and Characterization of Nanoparticles (NP) Engineered to Release Encapsulated Radionuclide Contents in the Tumor Interstitium and to Adhere Primarily to Tumor Extracellular Matrix (ECM) with Minimal Internalization by Cells

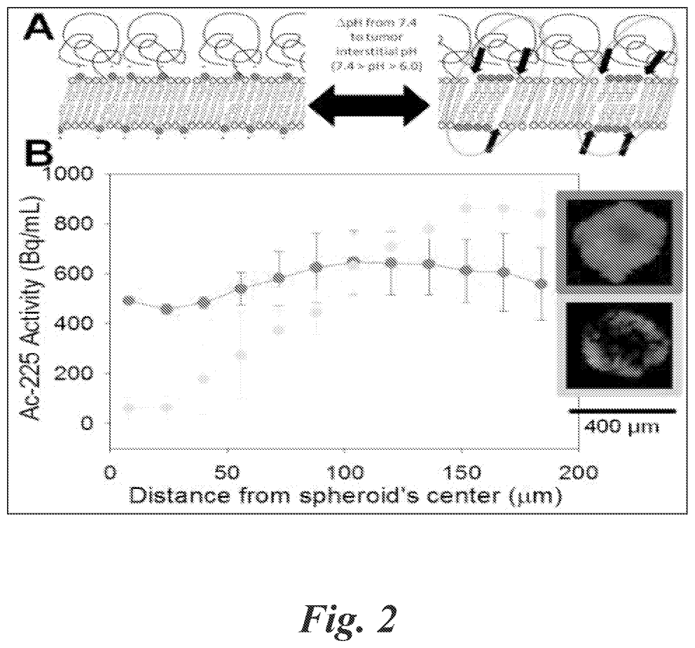

For the property of drug release from NP in the tumor interstitium, NP were designed which contain pH-responsive membranes forming phase-separated domains (resembling patches) with lowering pH (see FIG. 2A) (Bandekar et al, 2012; Karve et al., 2009; Karve et al., 2008; Karve et al., 2010). During circulation in the blood, these NP comprised well-mixed, uniform membranes and stably retained their encapsulated contents (FIG. 2A). In the acidic tumor interstitium, occurrence of membrane-phase separation resulted in the formation of “registered” patches that span the bilayer (FIG. 2A). Bandekar and Sofou, 2012. This membrane rearrangement was utilized to create pronounced grain boundaries around the patches, enabling release of the encapsulated therapeutic agents which then—in a drug delivery setting—diffuse deeper into solid tumors as is shown by their micro-distributions in spheroids (see FIG. 2B) (Zhu et al., 2017; Stras et al., 2016).

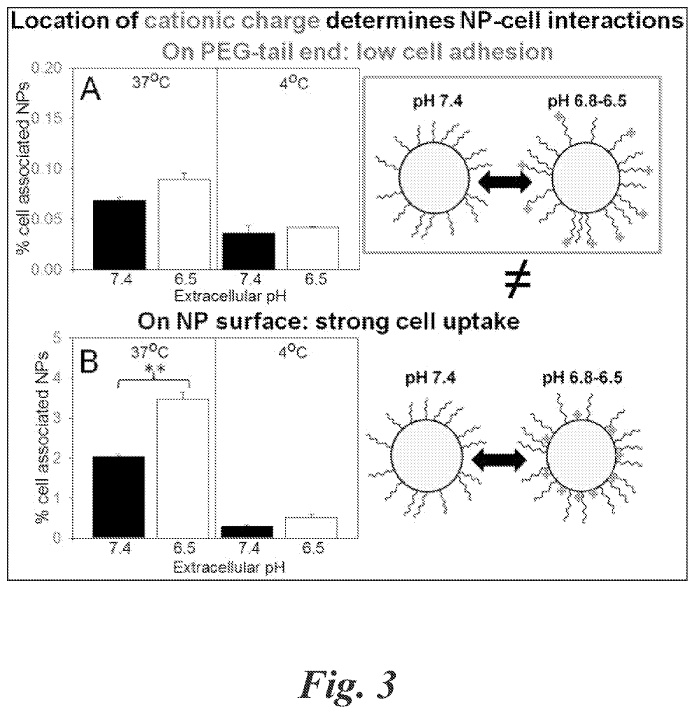

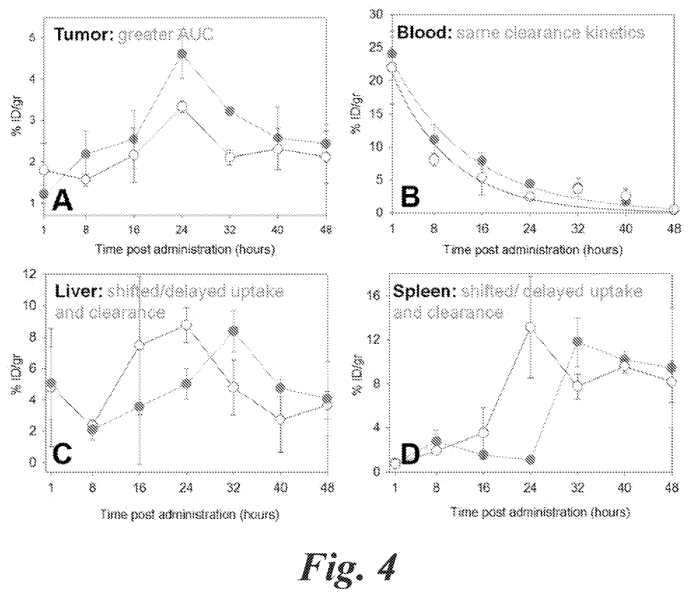

The property of NP adhesion to tumor ECM was achieved by attaching an “adhesion polymer” to the outer coronal of the NP in order to generate a positive charge (see FIG. 3A) which is ‘turned on’ in the slightly acidic pH of the tumor interstitium (pHₑ˜6.7-6.5) (Helmlinger et al., 1997; Vaupel et al., 1989). The adhesion polymer was generated by conjugating the moiety dimethyl ammonium propane (DAP) onto a free PEG-chain end, and the conjugated PEG-chains were grafted on NPs, with intrinsic pKa of ˜6.7 (Stras et al., 2020; Bailey and Cullis, 1994). NPs comprising the adhesion polymer (blue symbols) did not significantly interact with cancer cells (binding/internalization), as shown in FIG. 3A (top panel), and their cell association was an order of magnitude lower than the interactions observed for previously reported cationic NPs, Bailey and Cullis, 1997, which bear the charge directly on the surface of the NP membrane (Sokolova et al., 2013; Lin and Alexander-Katz, 2013) (FIG. 3B). Instead, NPs comprising the adhesion polymer adhere on the ECM of tumors, a property which seems to play a central role in the delayed clearance of NPs from tumors. Stras et al., 2020. Thus, the primary effect of the adhesion property is to increase tumor uptake of NPs and to delay NP clearance from tumors in vivo (FIG. 4A) without affecting the NP blood clearance kinetics (FIG. 4B).

NP were actively loaded, Zhu et al., 2017, with ²²⁵Ac using a calcium ionophore (A23187). The loading yields were 70-90% of introduced radioactivity, and the encapsulated ²²⁵Ac-DOTA was stably retained within NP in challenging conditions. The specific radioactivity (radioactivity per NP) is highly adjustable depending on the radioactivity levels used. The radioactive contents are triggered by the slightly acidic pHₑ in the tumor interstitium to be released from NP fast and extensively. Zhu et al., 2017.

### Example 2

### Combination of Two Carriers with Complementary Micro-Distributions of the Same α-Particle Emitter Results in Higher Efficacy

Balb/c nu/nu female mice with HER2+ BT474 breast cancer orthotopic xenografts and NOD SCID gamma male mice with PSMA+PC3-PIP prostate cancer xenografts were simultaneously administered the same total dose of the α-particle emitter Actinium-225 (²²⁵Ac) that was equally split between two carrier modalities: tumor responsive nanoparticles (²²⁵Ac-DOTA NP) and radiolabeled antibodies (²²⁵Ac-DOTA-SCN-Ab) targeting HER2 or PSMA. Animals were administered once I.V. with 80% of MTD (200 nCi and 150 nCi per 20 gr Balb/c and NSG mouse, respectively). No liver, spleen and/or kidney toxicities were observed.

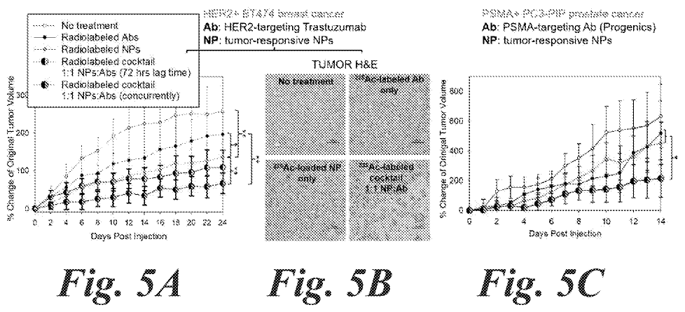

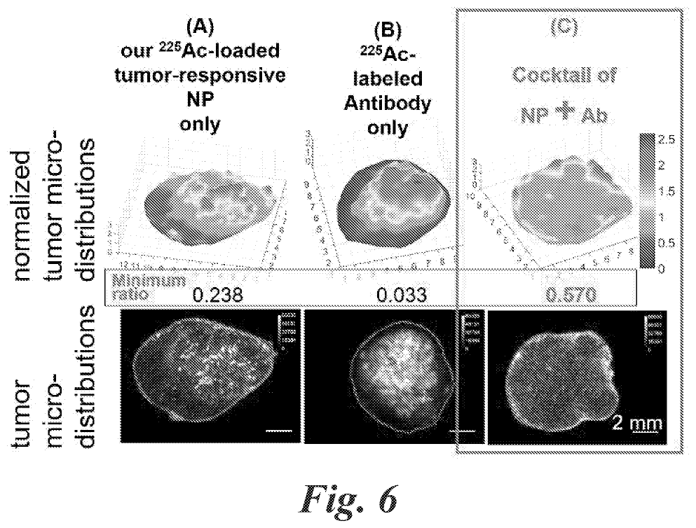

On both animal models, synergy in controlling tumor growth in vivo was observed, as shown in FIG. 5A, FIG. 5B, and FIG. 5C.

The delivery schedule of both carriers was varied in order (1) increase administered doses without increasing off-target toxicities and/or (2) further minimize of any off-target dose uptake for patients with already compromised functions of the breast or prostate. The radiolabeled Ab was administered 72 hours after the NP (²²⁵Ac-DOTA NP) to allow for clearance of the NP from the liver, which is also the main off-target organ for the Ab. Enhanced tumor growth control was still observed with this approach, which could also potentially reduce off-target toxicities, since the rate of total delivered dose at the liver was decreased.

Most nanoparticles (NP) are taken up by the liver and spleen, and antibodies are mainly taken up by the liver, making these sites the potentially dose-limiting normal organs. Although αRPT-induced toxicities have not been detected in mouse models, Prasad et al., 2021; Zhu et al., 2017, a range of lag times will be introduced into in the treatment schedule for mice, with the goal of minimizing potential toxicities at the critical off-target organs. First, the radiolabeled NP will be administered, and, following a lag time sufficient for NP clearance from the liver and spleen, the radiolabeled Ab will be administered (which also accumulates in the liver). In this manner, the rate of total delivered dose to the liver will dramatically decrease.

### Example 3

### In Vivo Uniform Micro-Distributions of ²²⁵Ac when Delivered by Tumor-Responsive Nanoparticles and Tumor-Targeting Antibodies

Tumors were extracted from the HER2+ cancers of the animals described in Example 1 24 hours post I.V. administration of ²²⁵Ac-DOTA NP, ²²⁵Ac-DOTA-SCN-Ab, or the cocktail containing the combination of ²²⁵Ac-DOTA NP and ²²⁵Ac-DOTA-SCN-Ab (NP+Ab). Tumor sections were imaged using an α-camera, which is a quantitative imaging technique developed to detect α-particles in tissues ex vivo (Back and Jacobsson, 2010). More uniform micro-distributions of ²²⁵Ac were observed when the ²²⁵Ac dose was split between the tumor-responsive NPs and the tumor-targeting antibodies as compared to compared to the same total dose delivered by either carrier alone (see FIG. 6A, FIG. 6B, and FIG. 6C).

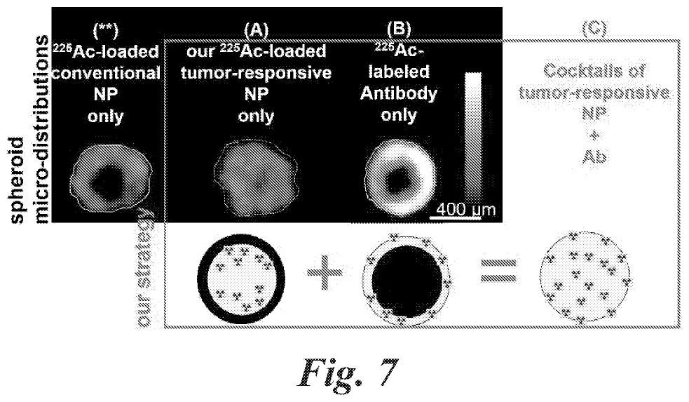

In vitro, the complementary micro-distributions of the two carriers were observed on sections of spheroids, which were used as surrogates of tumor avascular regions). In particular, the tumor-responsive NPs delivered lethal doses of ²²⁵Ac-DOTA in the deep areas of the tumor where antibodies do not reach, as show in FIG. 7A. In addition, ²²⁵Ac-labeled antibodies overkill the tumor periphery where the NP-based carrier is subject to fast clearance of released drugs, as shown in FIG. 7B. Conventional NP (non-releasing, non-adhering) exhibit low penetration of the α-particle emitters (FIG. 7 indicated with **).

Thus, the above approach can deliver a large number of α-particles at the tumor periphery where the cell number is greatest and where cells grow most aggressively, and, simultaneously, a high capacity penetrating payload to the tumor interior where the dormant and resistant cells are most likely to be responsible for treatment failure. This approach overcomes delivery obstacles while simultaneously reducing potential toxicity, primarily due to lower administered doses capable of efficiently inhibiting tumor growth.

### Example 4

### Tumor-Type Agnostic Inhibition of Growth of Cancers with Variable Expression of the Targeted Surface Marker

As an indirect surrogate of tumor recurrence, an outgrowth assay was performed on HER2-overexpressing BT474 breast cancer spheroids, and HER1-moderately expressing MDA-MB-231 triple negative breast cancer spheroids. Spheroids were treated with ²²⁵Ac-DOTA NP, ²²⁵Ac-DOTA-SCN-Ab, or the cocktail containing the combination of ²²⁵Ac-DOTA NP and ²²⁵Ac-DOTA-SCN-Ab (NP+Ab). Upon exposure to treatment (6 and 24 hours for NPs and Abs, respectively, to match their corresponding circulation half-lives in mice), spheroids were plated on adherent surfaces and cells were allowed to grow. The cells were counted when the cells from untreated spheroids reached confluency. The number of live cells was then reported as % outgrowth relative to the counted numbers of cells that received no treatment.

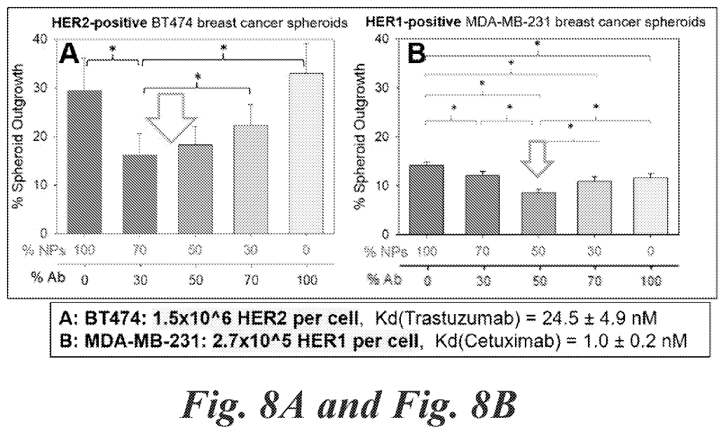

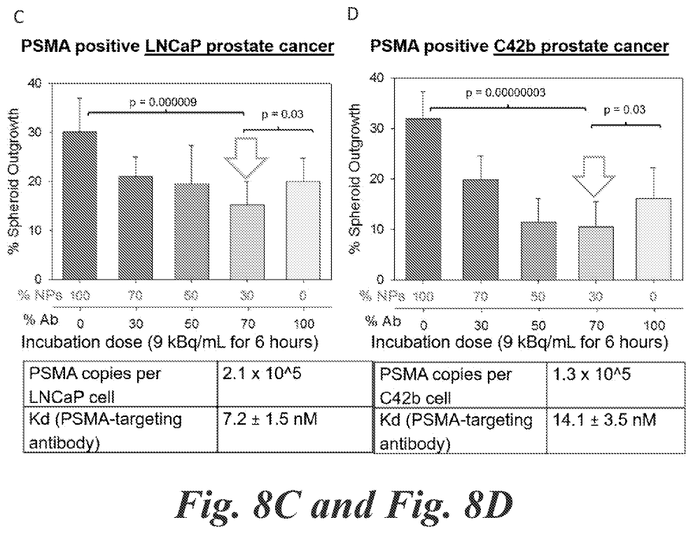

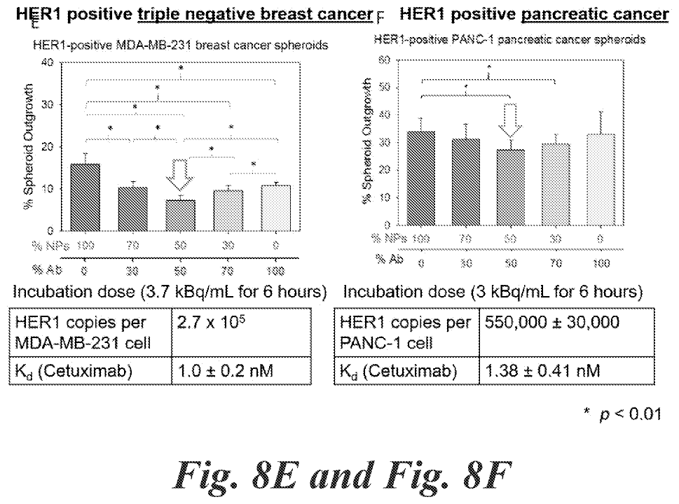

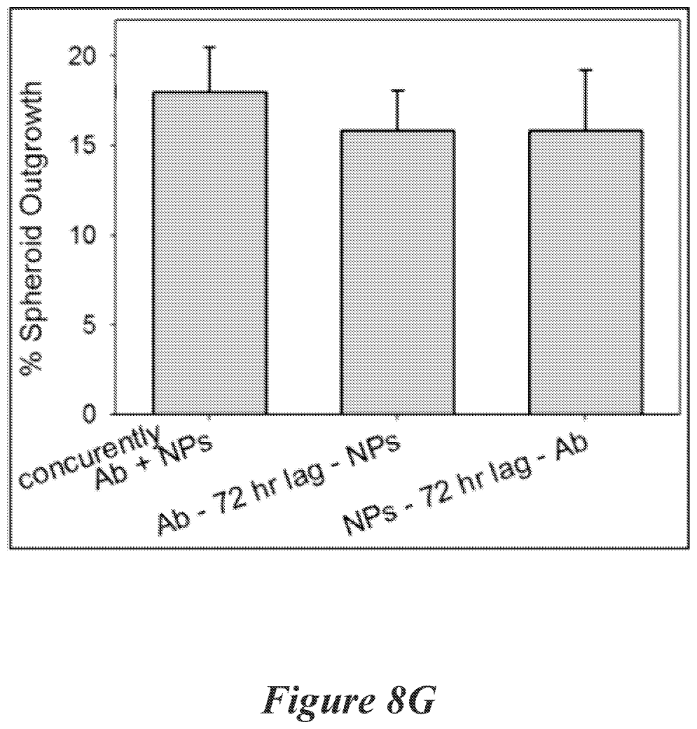

HER2-overexpressing BT474 breast cancer spheroids, and HER1-moderately expressing MDA-MB-231 triple negative breast cancer spheroids were each much better controlled when treated with the NP+Ab cocktail than when treated with either carrier alone, as shown in FIG. 8A and FIG. 8B. The results of this experiment also show that only the radiolabeled targeting antibody (Ab), and not the NP, needs to be tumor-type specific, further shown in FIG. 8C, FIG. 8D, FIG. 8E, FIG. 8F. As such, the methods described herein are tumor agnostic.

### Example 5

### Combination of Carriers with Complementary Intratumoral Microdistributions of Delivered Alpha Particles May Realize the Promise for Actinium-225 in Large Solid Tumors

### 5.1 Overview

Alpha-particle radiotherapy has already been shown to be impervious to most resistance mechanisms. An α-particle therapy effectively treating established (i.e., large, vascularized) soft-tissue lesions, as well as the smaller metastases is critical to successfully handling solid tumor patients at every stage of their disease. In large tumors, however, the diffusion-limited penetration depths of radiolabeled antibodies and/or nanocarriers (up to 50-80 μm) combined with the short range of α-particles (4- to 5-cell diameters) may result in only partial tumor irradiation potentially limiting treatment efficacy.

To address partial tumor irradiation in solid tumors, the presently disclosed strategy is grounded in the simultaneous delivery of the same emitter by combinations of carriers with complementary intratumoral microdistributions of the delivered α-particles. In some embodiments, the α-particle generator Actinium-225 (²²⁵Ac) is combined with (1) a tumor-responsive liposome that upon tumor uptake releases in the interstitium a highly-diffusing form of its radioactive payload ([²²⁵Ac]Ac-DOTA), which may penetrate the deeper parts of tumors where antibodies do not reach, with (2) a separately administered, less-penetrating radiolabeled-antibody irradiating the tumor perivascular regions from where liposome contents clear too fast.

On a murine model with orthotopic HER2-positive BT474 breast cancer xenografts, the biodistributions of each carrier were evaluated, and the control of tumor growth was monitored after administration of the same total radioactivity of ²²⁵Ac delivered (1) by the [²²⁵Ac]Ac-DOTA-encapsulating liposomes, (2) by the [²²⁵Ac]Ac-DOTA-SCN-labeled-Trastuzumab, and (3) by both carriers at equally split radioactivities.

The inhibition of tumor growth was significantly more pronounced when the same total radioactivity was divided between the tumor-responsive liposomes and the targeting radiolabeled-antibodies, as compared to the growth delay by the same total radioactivity when delivered by either of the carriers alone. This finding was attributed to the more uniform intratumoral microdistributions of α-particles imaged on tumor sections by an α-Camera.

This strategy provides strong evidence that combining carriers with complementary microdistributions of the delivered α-particles within established solid tumors may address the partial tumor irradiation that could challenge efficacy.

### 5.2 Background

Metastatic and/or recurrent solid cancers present an all too common clinical challenge partly due to development of resistance. American Cancer Society, 2020. Clinical studies with α-particle emitters have sometimes had exceptional outcomes on patients with metastatic prostate cancer resistant to approved options. Kratochwil et al., 2016. Success of α-particle radiotherapy against solid, soft-tissue cancers, however, has been confined to the treatment of disseminated, relatively small metastases. Navarro-Teulon et al., 2013. A treatment against established (i.e., large, vascularized) lesions in conjunction with a treatment of smaller metastases is critical to successfully handling solid tumor patients at every stage of their disease.

Alpha-particle radiotherapy has been shown to be impervious to most resistance mechanisms. Yard et al., 2019. The complexity and level of double-strand DNA damage caused by only a few tracks of α-particles across the cell nucleus, Humm and Chin, 1993; Macklis et al., 1988, overwhelms cellular repair mechanisms mostly independently of the cell-oxygenation state and cell-cycle, McDevitt et al., 2018; this inability to repair lethal damage is the reason that α-particle therapy, if optimally delivered, is impervious to resistance. The short range of α-particles (40-100 μm), which is ideal for localized irradiation and minimal irradiation of surrounding healthy tissues, also limits penetration within large tumors; the diffusion-limited penetration depths of radiolabeled antibodies and/or nanocarriers (up to 50-80 μm) combined with the short range of α-particles may result in only partial tumor irradiation. Zhu et al., 2017. Importantly, partial tumor irradiation may limit the treatment efficacy of α-particle therapies irrespective of any augmenting by-stander effects. Wang and Coderre, 2005.

Tumor-selective delivery strategies for α-particle therapies that aim to spread the intratumoral α-particle distributions over larger regions within solid tumors, and to prolong exposure of cancer cells to delivered radiotherapeutics, may improve efficacy against established tumors. Toward this goal, the presently disclosed subject matter provides a strategy to deliver the α-particle generator Actinium-225 (²²⁵Ac) as uniformly as possible throughout established tumors using a HER2-positive human breast cancer, chosen as a model tumor for proof-of-concept. More particularly, in some embodiments, two different delivery carriers of ²²⁵Ac were combined: tumor-responsive liposomes and HER2-targeting antibodies, each administered separately. The liposomes were engineered to have two key properties for the implementation of the presently strategy: (1) to clear slowly from tumors and, (2) only in the tumor interstitium, to release highly diffusing forms (due to their small size) of the α-particle emitters ([²²⁵Ac]Ac-DOTA) which then may penetrate in the deep parts of tumors, where antibodies do not reach. Zhu et al., 2017; Thurber et al., 2007. The antibodies also were labeled with ²²⁵Ac, which they deliver mostly closer to the tumor periphery (the perivascular regions), where the liposome-based modality suffers due to fast clearance of released therapeutic agents. Stras et al., 2020.

The presently disclosed tumor-responsive liposomes were designed (and previously demonstrated) to exhibit the following properties, all of which were triggered by the slightly acidic pH in the tumor interstitium (extracellular pH, pHₑ˜6.7-6.5), Vaupel et al., 1989: (1) adherence to the tumors' extracellular matrix (ECM) (resulting in slower liposome clearance from the tumor), Stras et al., 2020; (2) low uptake and/or internalization by cancer cells, Stras et al., 2020; and, (3) release of contents directly in the interstitium triggered by the tumor acidity. Zhu et al., 2017. The HER2-targeting antibody, Trastuzumab, that was administered separately from liposomes, was chosen because of its high affinity for the HER2 receptor, reasonable radiolabeling and well-characterized in vivo behavior.

Without wishing to be bound to any one particular theory, it is thought that the combination of different carriers that deliver α-particle radiotherapies to complementary regions of the same solid tumor result in more uniform irradiation over a larger fraction of the solid tumor's volume and, therefore, in greater tumor growth inhibition compared to the same administered radioactivity delivered by each carrier alone.

### 5.3 Materials and Methods

### 5.3.1 Materials

All lipids including 1,2-distearoyl-sn-glycero-3-phosphoethanolamine-N-PEG2000-dimenthylammonium propanoyl (DSPE-PEG-DAP, the ‘adhesion’ lipid) were purchased from Avanti Polar lipids (Alabaster, Ala.). 1,4,7,10-tetraazacyclododecane-1,4,7,10-tetraacetic acid (DOTA) and p-SCN-Bn-DOTA (DOTA-SCN) were purchased from Macrocyclics (Plano, Tex.). AlexaFluor 647-NHS-Ester and CFDA-SE (carboxyfluorescein diacetate succinimidyl ester) were purchased from ThermoFisher. Trastuzumab was purified from Herceptin® which was a gift from Genentech (South San Francisco, Calif.). Actinium-225 (²²⁵Ac, actinium chloride) was supplied by the U.S. Department of Energy Isotope Program, managed by the Office of Isotope R&D and Production.

### 5.3.2 Liposome Formation and Characterization

Tumor-responsive liposomes were formed using the thin-film hydration method as described in detail in Zhu et al., 2017. Liposomes were characterized for size and zeta potential using a Zetasizer NanoZS90 (Malvern, United Kingdom).

5.3.3 Radiolabeling of carriers with ²²⁵Ac/¹¹¹In

DOTA-SCN-Trastuzumab (and/or DTPA-SCN-Trastuzumab) was radiolabeled and characterized for purity, stability and immunoreactivity as described in Supporting Information and previously reported. Zhu et al., 2017. Liposomes encapsulating DOTA (or DTPA) were actively loaded with ²²⁵Ac (or ¹¹¹In) using the ionophore A23187. Zhu et al., 2017.

### 5.3.4 Cell Culture

The cell lines BT474 and the Trastuzumab resistant BT474 (BT474-R) were obtained from ATCC and were grown in cell culture treated flasks at 37° C. and 5% CO₂ in Hybricare media buffered with sodium bicarbonate supplemented with 10% FBS, 100-U/mL penicillin and 100-mg/mL streptomycin.

### 5.3.5 Clonogenic Survival

After incubation for 6 hours of cell monolayers with varying concentrations of radioactivity, the cells were washed and were plated in dishes to grow until formation of colonies, as described in detail in Prasad et al., 2021.

### 5.3.6 Spheroid Formation and Spatiotemporal Profiles in Spheroids

BT474 cells were seeded on polyHEMA-coated, 96-well round-bottomed plates, were centrifuged, and were allowed to grow to reported size before initiation of treatment. Zhu et al., 2017. Spheroids were incubated with liposomes (containing fluorescently labeled lipids and encapsulating a hydrophilic fluorophore as drug surrogate) or with the fluorescently labeled antibody. As described in Zhu et al., 2017, and previously reported, Stras et al., 2020, at different times spheroids were sampled, sliced and the equatorial section was imaged and analyzed using an eroding code. The spatial distributions at each time point were integrated (using the trapezoid rule) to evaluate the time-integrated-concentration(s) at each radial position.

### 5.3.7 Spheroid Growth and Outgrowth Studies

Spheroids were incubated for 6 hours with [²²⁵Ac]Ac-DOTA-loaded liposomes (1-mM total lipid) and/or 24 hours with [²²⁵Ac]Ac-DOTA-SCN-Trsastuzumab (10 μg/mL). Upon completion of incubation, spheroids were transferred to fresh media and the spheroid volume was monitored until the non-treated spheroids stopped growing (17 days later) at which point spheroids were individually plated on cell culture treated, flat-bottom 96-well plates and were allowed to grow. The number of live cells per well was reported as % outgrowth relative to the numbers of live cells that received no treatment, when the latter reached confluency.

### 5.3.8 Animal Studies

One million BT474 cells suspended in 100 μl of 50:50 v:v Matrigel™:serum-free Hybricare media were inoculated into the second mammary fat pad of 5-to-6 week old NCR-nu/nu female mice (Taconic, Germantown, N.Y.) at 24 hours following subcutaneous implantation of a 17β-estradiol (1.7 mg)+progesterone (10 mg) hormone pellet (Innovative Research of America, Sarasota, Fla.).

Upon tumors reaching 50 mm³, mice were randomly assigned to a group. For biodistribution studies, animals were I.V. administered (352-444 kBq, 9.5-12 μCi, per animal) of [¹¹¹In]In-DTPA-encapsulating liposomes or [¹¹¹In]In-DTPA-SCN-Trastuzumab in 0.1 mL, and at different time points, animals were sacrificed, and organs were weighed and measured for radioactivity. In addition to the cold conditions and to no treatment, for treatment studies, mice were administered I.V. a single 0.1 mL injection of 4.625 kBq (125 nCi) or 9.25 kBq (250 nCi) of [²²⁵Ac]Ac-DOTA-SCN-Trastuzumab, [²²⁵Ac]Ac-DOTA-loaded liposomes, or a combination of the two at constant total administered radioactivity. The total mass of antibody was kept constant at 15 μg/mouse. Every other day, mice were weighed, and tumor volumes were measured with a digital caliper (resolution 0.01 mm).

### 5.3.9 α-Camera Imaging

Tumor-bearing mice were injected I.V. with [²²⁵Ac]Ac-DOTA-SCN-labeled antibody, [²²⁵Ac]Ac-DOTA-loaded liposomes or both at 148 kBq (4 μCi) total radioactivity and were sacrificed 24 hours later. Tumor and tissues were immediately harvested, embedded in OCT compound, frozen and then cryosectioned. The exposure time for the α-Camera was 24 hours per sample, Black and Jacobsson, 2010, and the images were analyzed using ImageJ 1.49b (NIH, Bethesda, Md.) after being decay-corrected to the time of sacrifice.

### 5.3.10 MRI Imaging for Evaluation of Tumor pHₑ

The method for evaluation of the tumor pHₑ maps, Pacheco-Torres et al., 2015, is described in detail in Prasad et al., 2021.

### 5.3.11 Statistical Analysis

Results are reported as the arithmetic mean of n independent measurements±the standard deviation. Significance in multiple comparisons and pair comparisons was evaluated by one-way ANOVA and unpaired Student's t-test, respectively, with p-values 0.05 considered to be significant.

### 5.4.1 Results

### 5.4.1.1 Carrier Characterization

Table 1A shows the change of liposomes' apparent zeta potential with lowering pH toward less negative values; this observation was partly attributed to the protonation of DAP, the ‘adhesion lipid’, with apparent pKa of 6.8, which was attached to the free end of PEGylated lipids. Stras et al., 2020. Acidification also resulted in release of encapsulated [²²⁵Ac]Ac-DOTA from liposome. Prasad et al, 2021. Characterization of the radiolabeled Trastuzumab is summarized in Table 1B.

TABLE 1A and TABLE 1B. Characterization of ²²⁵Ac-labeled (TABLE 1A) tumor-responsive liposomes and (TABLE 1B) the HER2-targeting Trastuzumab. **indicates 0.001<p-values<0.01; ***p-values<0.001.

TABLE 1B

| Radiolabeling | | Specific activity | Radiochemical | 24 hour | K_{D} (nM) | |
|---|---|---|---|---|---|---|
| efficiency % | Immunoreactivity % | (MBq/mg Ab) | Purity | retention % | BT474 | BT474-R |
| 53.8 ± 11.4 | 95.9 ± 1.4 | 3.4 ± 0.7 | 97.8 ± 1.8 | 90.6 ± 2.5 | 24.6 ± 4.9 | 10.2 ± 1.7 |
| n = 14 | n = 14 | n = 11 | n = 14 | n = 14 | | |
| | | starting activity | | | | |
| | | 0.4-1.1 MBq | | | | |

### 5.4.1.2 Survival Assay

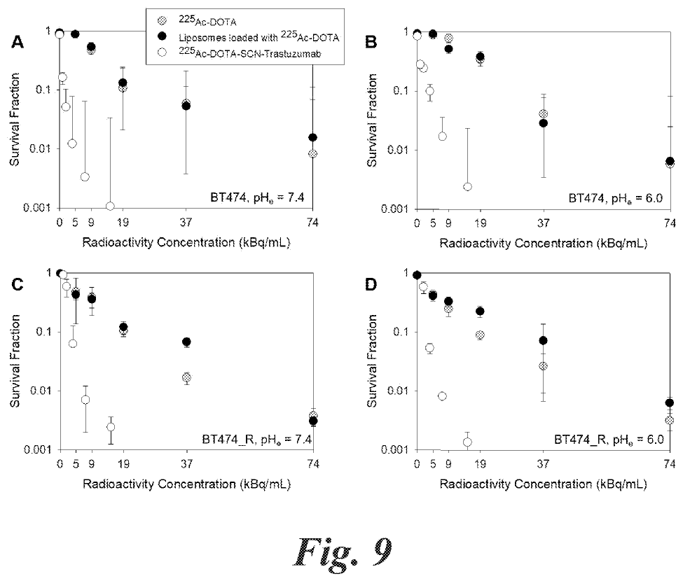

Both cell lines, in monolayers, exhibited same sensitivity to free [²²⁵Ac]Ac-DOTA and to [²²⁵Ac]Ac-DOTA-encapsulating liposomes, independent of pH (FIG. 9). This characteristic was expected, since liposomes were designed to minimally associate with cancer cells, Stras et al., 2020, as also is the case for free [²²⁵Ac]Ac-DOTA. Both cell lines exhibited comparable survival responses to [²²⁵Ac]Ac-DOTA-SCN-Trastuzumab demonstrating lack of resistance to α-particles independent of the reported resistance to Trastuzumab for BT474-R. The HER2 expression levels by the two cell lines were comparable (1.5×10⁶ vs 0.93×10⁶ copies per cell.

### 5.4.1.3 Spheroids: Spatiotemporal Carrier Microdistributions and Response to Delivered ²²⁵Ac

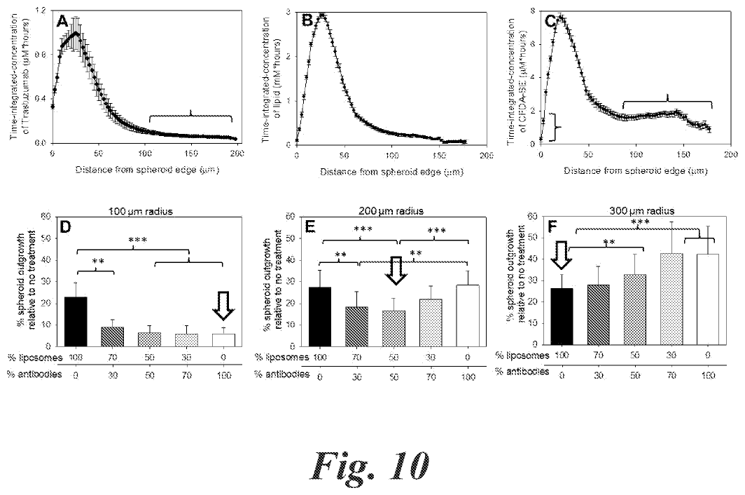

The time integrated microdistributions of Trastuzumab in spheroids (FIG. 10A), used as surrogates of tumor avascular regions, exhibited high accumulation only within the first 60 μm from the spheroid edge with less than 10% of the peak value at distances beyond 100 μm from the edge (indicated by a horizontal bracket). As expected, liposomes did not penetrate the spheroids longer distances than the antibody (FIG. 10B). Conversely, at distances 80 μm from the spheroid edge and beyond, the fluorophore (FIG. 10C), that was used as a drug surrogate and was released from the liposomes, exhibited uniform time-integrated values at approximately 25% of its peak value (indicated by a horizontal bracket). Close to the spheroid edge, however, the released fluorophore cleared too fast from the spheroid (indicated by the vertical bracket). The acidification of the spheroids' interstitial pH (pHₑ), which triggers the properties of liposomes' ECM-adhesion and content release, ranged from 7.4 close to the spheroids' edge to around 6.5 at the spheroid center, Prasad et al, 2021.

Following exposure to ²²⁵Ac, divided between liposomes and the antibody, the carrier(s) resulting in greatest suppression of spheroid outgrowth depended on the spheroid size at the time of treatment. On small spheroids (100-μm radius), delivery of radioactivity by the targeting antibody (FIG. 10D) was most efficient. The heterogenous distribution of Trastuzumab (high uptake but mostly localized close to the spheroid edge, FIG. 10A) did not impact efficacy because the longest spheroid distance (100-μm radius) was comparable to the range of α-particles in tissue (<100 μm). Zhu et al., 2017. On large spheroids (300-μm radius, corresponding to avascular distances almost 3 times longer than the range of α-particles in tissue) delivery of radioactivity by liposomes, that released in the interstitium [²²⁵Ac]Ac-DOTA, was most efficient; this was attributed to the deeper penetration (as supported by the fluorescent surrogate in FIG. 10C) of released [²²⁵Ac]Ac-DOTA toward the spheroid center. Zhu et al., 2017. In spheroids with intermediate size (200-μm radius), the combination of the two carriers resulted in better cell kill.

### 5.4.1.4 Tumor pHₑ Measurement

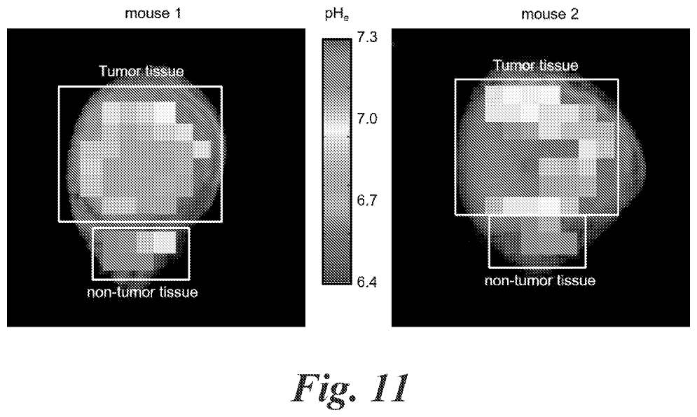

The MRI-acquired tumor- and tissue-pHₑ maps shown on FIG. 11 confirmed the tumor interstitial acidity that locally reached pHₑ values adequate to trigger both the release and adhesion properties of liposomes. Notable was the heterogeneity of pHₑ maps within tumors and between the two animals.

### X.4.1.5 In Vivo Assessment

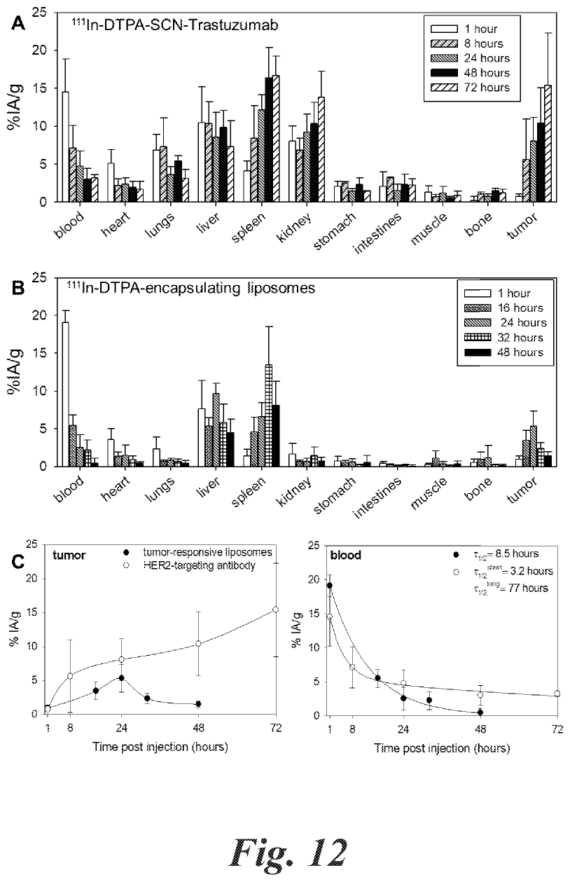

The biodistributions demonstrated the significantly greater mean tumor uptake of injected radioactivity when delivered by Trastuzumab compared to the tumor uptake when radioactivity was delivered by liposomes (FIG. 12C). Indium-111 was used as surrogate of ²²⁵Ac, not of its daughters. This choice was based on previous studies demonstrating that ¹¹¹In-labeled Trastuzumab and ²²⁵Ac-labeled Trastuzumab resulted in similar biodistributions. Borchardt et al., 2003. In liposomes, the same retention of ¹¹¹In and ²²⁵Ac by tumor-responsive liposomes, justified the use of ¹¹¹In as surrogate of ²²⁵Ac. Prasad et al., 2021. Lastly, for the cell-internalizing Trastuzumab, since ²²⁵Ac daughters are generally not expected to significantly translocate from the site of the original parent decay, Behling et al., 2016, tracking of ¹¹¹In as surrogate for the parent ²²⁵Ac nucleus would be also informative of the projected biodistributions of its daughters. For non-internalizing carriers, as are the liposomes, the fate of the longest-lived daughter, Bismuth-213, is well understood and rapidly localizes to the kidneys. Josefsson et al., 2018.

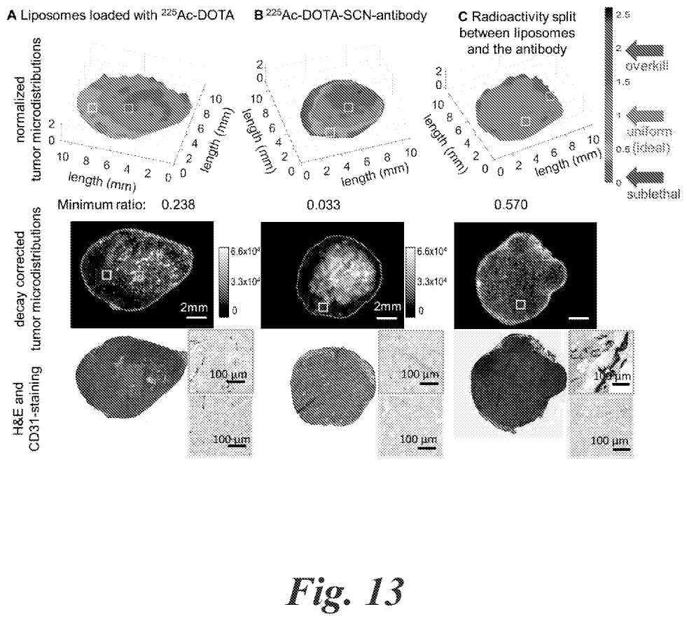

As expected from the spheroid studies, the α-Camera imaged microdistributions of ²²⁵Ac in tumor sections, were more heterogeneous when the entire radioactivity was delivered by each carrier alone (FIG. 13A and FIG. 13B) compared to the simultaneous delivery of the same total radioactivity that was split between the two carriers (FIG. 13C). The top panel shows the normalized tumor microdistributions (where each pixel intensity was divided by the average of the intensities over the entire tumor section), and areas colored in red (ratio equal to 1) indicated local values closer to the mean tumor-delivered radioactivities. Importantly, in both tumor sections where ²²⁵Ac was delivered only by a single carrier, the cyan and deep blue colored regions occupied significant area fractions; dark blue regions indicated local delivered radioactivities well-below the mean tumor-delivered values and, therefore, could result in lower cell kill. In densely vascularized areas (CD31-positive areas, green inserts) the delivered radioactivity levels were higher than in sparsely vascularized areas (yellow inserts).

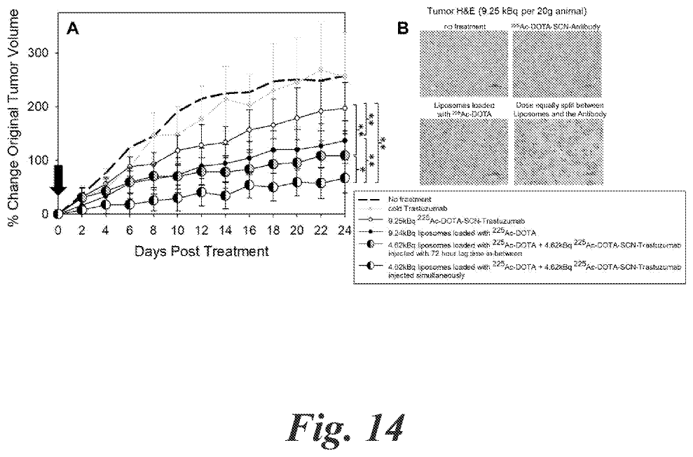

In agreement with the extent of uniformity in tumor microdistributions of ²²⁵Ac, the greatest inhibition of the volume growth of orthotopic BT474 xenografts was observed when radioactivity was delivered by equally splitting the same total radioactivity (9.25 kBq (250 nCi) (Zhu et al., 2017)) between the two carriers (4.62 kBq+4.62 kBq) that were administered simultaneously (half-black-half-white circles); this is to be contrasted to administering the same total radioactivity (9.25 kBq) by each carrier alone (p-values <0.001, FIG. 14A). Notably, based on the biodistributions on FIG. 12, the equal radioactivity split between the two carriers that resulted in best tumor inhibition would be expected to deliver less radioactivity per tumor mass than when the antibody alone was used, underscoring the significance of α-particle microdistributions within solid tumors.

Pathology evaluation of tumors on day 24 demonstrated visibly increased collagen upon treatment with ²²⁵Ac when delivered by the combination of both carriers compared to each carrier alone (FIG. 14B). Histopathology analysis of H&E stained sections of organs showed no noteworthy hepatic, cardiac, or renal toxicities across all constructs at the time of sacrifice. Long-term toxicities, evaluated 9.5 months post I.V. injection, on NSG mice treated at the MTD with the liposomal forms of ²²⁵Ac have not shown renal toxicities. Prasad et al., 2021. Slight inflammation in the diaphragm of the liposome-only treatment group was observed, but otherwise there was no visible lung inflammation. Additionally, increased cell death in and reduced size of the spleen was observed in the liposome-only condition, in agreement with their significant splenic uptake. The animal weight during the study did not decrease below 10% of the weight at the initiation of treatment.

Survival was not a meaningful end point in this study, because tumor growth was estrogen dependent; following a single estrogen pellet implantation, tumor growth rates (as shown by the non-treatment animal group, FIG. 12A) reached an asymptote after approximately 60 days from implantation.

### 5.5 Discussion

We hypothesized that improvement of the spatial uniformity of an α-particle emitter within solid tumors may address the challenge of partial irradiation by α-particles and, therefore, improve efficacy. In this study we demonstrated, using a simple and clinically implementable approach, that improvement of the spatial intratumoral uniformity of ²²⁵Ac can be enabled by combinations of two separate carriers with complementary tumor microdistributions. Our approach optimized payload delivery; delivering a large number of α-particles at the tumor perivascular regions (via the targeting antibody) where the cell number is greatest and where cells are growing most aggressively, and, simultaneously, a high capacity penetrating payload to the tumor interior (via the tumor-responsive liposomes) where the dormant and resistant cells are most likely to be responsible for treatment failure. Vaupel, 2004. In the present study the same total administered radioactivity was equally divided between the two carriers resulting in synergistic inhibition of tumor growth compared to each carrier alone. It is possible, and this is currently under investigation, that different radioactivity split ratios between the two carriers may result in even better tumor growth inhibition solely due to more uniform spatiotemporal microdistributions of emitters within tumors.

Two points are key to the clinical relevance and applicability of this approach; first is the vascular permeability of tumors to the administered liposomes; not all human established metastases exhibit vascular permeability to liposomes, but when they do, then the extent of liposome uptake by tumors is strongly and favorably correlated with tumor response, as has been clinically proven. Lee et al., 2017. Second is the acidification of the intratumoral pHₑ as it relates to triggering the properties of adhesion and release on our liposomes; acidification is common on tumors of patients with breast (and other) cancers, Vaupel, 2004, and is correlated with highly aggressive tumors. Vaupel et al., 1989; Vaupel, 2004; Estrella et al., 2013. The tumor pHₑ values reported in human tumors 6.60-6.98, Vaupel et al., 1989, are comparable to the values measured on the animal model used herein.

Regarding toxicities, the liver and spleen were the common off-target organs for both the liposomes and the antibody delivering ²²⁵Ac. Hepatic toxicity was not observed in the present study where 63% of the MTD, Zhu et al., 2017, was administered. Interestingly, the patterns of liver irradiation by each carrier, expected to affect liver toxicity, were different: the uniform liver infiltration, and irradiation, by the radiolabeled Trastuzumab was in striking contrast to the ‘grainy’ liver irradiation by liposomes. Prasad et al, 2021. A mechanism which could explain the latter finding is liposomes' sequestration by Kupffer cells, that reside on the luminal side of the liver sinusoids, possibly limiting irradiation of hematocytes. Regarding the spleen, we have previously shown that at the MTD of ²²⁵Ac delivered by liposomes, the initial splenic moderate-to-high hemosiderin deposition in the red pulp and reduced extramedullary hematopoiesis, observed right after administration of radioactivity, was found to subside and the spleen to fully recover after 9.5 months on tumor free mice. Prasad et al., 2021.

Specific to ²²⁵Ac are renal toxicities in mice which were previously partially connected to the escape in the blood of the last radioactive daughter of ²²⁵Ac, Bismuth-213, when ²²⁵Ac was delivered by long circulating carriers, such as antibodies, in addition to antibody renal uptake. Schwartz et al., 2011. Long-term toxicity studies of the radiolabeled Trastuzumab at 63% of the MTD have not been performed, at which the current therapeutic study was assessed, but approaches to address renal toxicities in mice are available. Jaggi et al., 2005. Regarding the other carrier, the ²²⁵Ac-encapsulating liposomes, renal toxicities at the MTD were not observed in tumor-free mice even 9.5 months post-administration. Prasad et al., 2021. In human trials using ²²⁵Ac-labeled antibodies or small molecules, Jurcic, 2020; Kratochwil et al., 2020, renal toxicities have not been reported yet. For the liposomal carriers, it would most possibly be the hepatic and splenic uptake which could raise concerns possibly requiring further investigation. Lee et al., 2017.

An observation that could potentially mitigate toxicities at the liver and spleen was that in spheroids, the order and lag time (up to 72 hours) between introduction of each of the two carriers did not significantly affect their collective inhibition of spheroid outgrowth (FIG. 8G). Translation of this observation in vivo, and administration of the radiolabeled antibodies 72 hours after administration of radiolabeled liposomes and liposome clearance from the liver and spleen (FIG. 12B), could reduce the delivered dose rates at the two-common off-target organs. FIG. 14A (half-black half-grey symbols) shows that improvement in tumor growth inhibition (relative to each carrier alone) was retained by the combined carriers even when the second half-dose by the radiolabeled antibodies was administered 72 hours after the first half-dose by radiolabeled liposomes. Interrogation of this approach, may ultimately, minimize any off-target effects on patients with already compromised functions of these organs.

### 5.6 Summary

The present disclosure investigated whether the partial irradiation of solid tumors by α-particles delivered with traditional radionuclide carriers be rectified to improve efficacy? These findings, in part, demonstrate that the partial irradiation of solid tumors by α-particle emitters can be rectified by combining carriers with complementary intratumoral microdistributions of the delivered α-particle emitters. The findings of this study show the potential to expand the impact of α-particle therapies to large solid tumors by choosing combinations of carriers based on the complementarity of the intratumoral microdistributions of the delivered α-particle emitters while maintaining the same administered radioactivities. With regard to patient care, the combination of separate carriers with complementary intratumoral microdistributions of α-particle emitters (²²⁵Ac in this study) could be a general strategy to control solid tumor growth both in preclinical investigations and in the design of personalized, α-particle therapies for patients.

### Example 6

### Therapeutic Strategy for Unresectable Large Solid Tumors

The presently disclosed subject matter provides a novel therapeutic strategy for unresectable large solid tumors. Existing therapeutic approaches are largely ineffective and fail for two major reasons: (1) the development of drug resistance, and (2) the inability to uniformly expose all malignant cells in a tumor to therapeutics at sufficient levels to cause cell death. Large, soft-tissue solid tumors are particularly challenging: cells in deep tumor regions far from vasculature often do not receive sufficient concentrations of therapeutics injected in the blood.

In some embodiments, the presently disclosed subject matter treats such tumors using alpha-particles delivered in a unique way that can uniformly treat large tumors. Alpha-particles (α-particles) are high energy, short range particles (travelling in tissue up to 4- to 5-cell diameters) emitted from radionuclides. The particles physically break DNA molecules as they traverse the cell nucleus. The inability to repair this DNA damage is the reason that α-particles, McDevitt et al., 2018, are impervious to most resistance mechanisms. Sgouros, 2019; Yard et al., 2019. The growing interest in α-particle emitters for cancer therapy is demonstrated by the recent FDA approval of [²²³Ra]RaCl₂ (Xofigo) for the treatment of bone metastases in prostate cancer, and the increasing list of clinical trials including targeted α-particle therapies of Actinium-225, ²²⁵Ac, a powerful emitter that also has been used by the inventors. Poty et al., 2018. To address the growing medical need for ²²⁵Ac and other α-particle emitters, three US National Labs are coordinating to evaluate alternative production methods, as is Triumph in Canada, and several private companies.

Alpha particles, however, have not been successful in treating patients with large tumors: the short range of α-particles combined with the short penetration from the vasculature of antibodies and nanoparticles, Zhu et al., 2017, result in only partial tumor irradiation. In contrast, the presently disclosed delivery strategy uniformly distributes α-particles within large solid tumors by simultaneously delivering the same α-particle emitter by different carriers, each killing a different region of the tumor: (1) a tumor-responsive lipid nanoparticle (NP) that upon tumor uptake releases in the interstitium a highly-diffusing form of its radioactive payload (²²⁵Ac-DOTA), which penetrates the deeper parts of tumors where antibodies do not reach, and (2) a separately administered, less-penetrating radiolabeled-antibody irradiating the tumor perivascular regions from where the NP's contents clear too fast.

The presently disclosed NPs are liposomes composed of lipid membranes forming phase-separated lipid domains (resembling lipid patches) with lowering pH. During circulation in the blood, such NPs comprise well-mixed, uniform membranes and stably retain their encapsulated contents. In the acidic tumor interstitium, lipid-phase separation results in formation of lipid patches that span the bilayer, creating transient lipid-packing defects along the patch boundaries, and enabling release of encapsulated agents. Zhu et al., 2017; Stras et al., 2020; Bandekar and Sofou, 2012. The NPs also have an adhesive property that enables NPs to bind to the tumors' extracellular matrix (ECM) delaying their clearance from tumors.

The presently disclosed approach overcomes delivery obstacles while simultaneously reducing toxicities due to the low administered doses necessary to effectively inhibit tumor growth. The NPs and the antibodies delivering radiotherapy are administered in the blood (either as IV or intra-arterially via a catheter) and are preferentially accumulating in tumors when the tumor vasculature is permeable to them (EPR effect) with minimal uptake to other normal tissues. Lee et al., 2017.

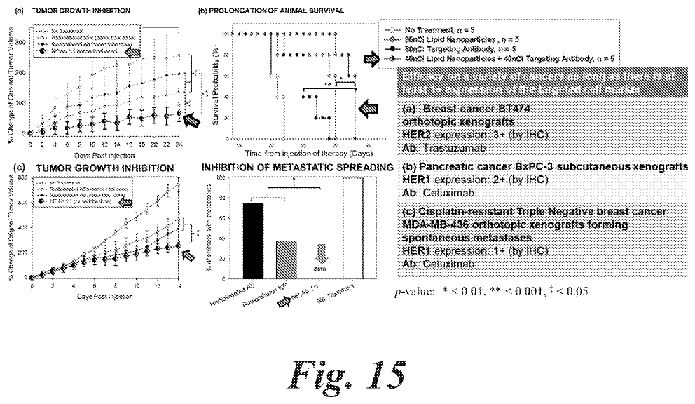

Further, the presently disclosed delivery strategy is tumor agnostic. The NPs are the same for all tumor types and have two key properties: (1) the release property and (2) the adhesion property. The choice of the antibody is determined by the type of receptors (that need not necessarily be overexpressed) on cancer cells. In some embodiments, the presently disclosed subject matter demonstrates that at 1+ receptor expression by cancer cells, the approach delivers lethal doses in the tumor's perivascular regions (FIG. 15).

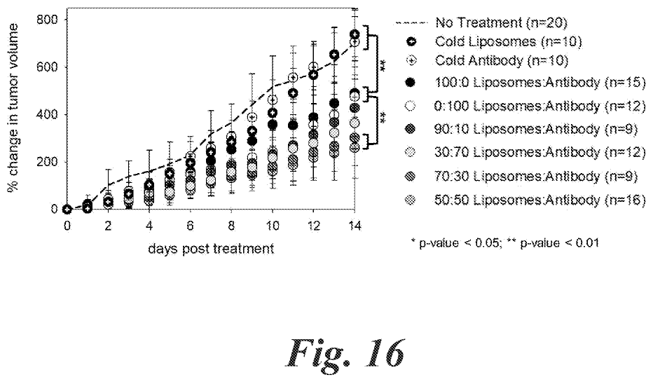

More particularly, in some embodiments, the presently disclosed subject matter provides a uniform and prolonged irradiation enabled by the unique delivery strategy (see, e.g., FIG. 13) that enables synergistic inhibition of tumor growth in a variety of human xenografts on mice (FIG. 15 and FIG. 16).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
American Cancer Society. Cancer Facts & Figures. 2020. www.cancer.org/research/cancer-facts-statistics.html.
Prasad A, Nair R, Bhatavdekar O, et al. Transport-oriented engineering of liposomes for delivery of α-particle radiotherapy: inhibition of solid tumor progression and onset delay of spontaneous metastases under review. 2021.
Prasad, A, Nair R, Bhatavdekar, O, Howe A, Salerno D, Sempkowski M, Josefsson A, Pacheco-Torres J, Bhujwalla Z, Gabrielson K L, Sgouros G, Sofou S, Transport-oriented engineering of liposomes for delivery of α-particle radiotherapy: inhibition of solid tumor progression and onset delay of spontaneous metastases, in press, European Journal of Nuclear Medicine and Molecular Imaging, 2021. https://doi.org/10.1007/s00259-021-05406-z
Stras S, Howe A, Prasad A, Salerno D, Bhatavdekar O, Sofou S. Growth of Metastatic Triple-Negative Breast Cancer Is Inhibited by Deep Tumor-Penetrating and Slow Tumor-Clearing Chemotherapy: The Case of Tumor-Adhering Liposomes with Interstitial Drug Release. Molecular Pharmaceutics. 2020; 17(1):118-31.
Pacheco-Torres J, Mukherjee N, Walko M, López-Larrubia P, Ballesteros P, Cerdan S, Kocer A. Image guided drug release from pH-sensitive Ion channel-functionalized stealth liposomes into an in vivo glioblastoma model. Nanomedicine: Nanotechnology, Biology and Medicine 2015; 11:1345-54.
Zhu C, Sempkowski M, Holleran T, Linz T, Bertalan T, Josefsson A, Bruchertseifer F, Morgenstern A, Sofou S. Alpha-particle radiotherapy: For large solid tumors diffusion trumps targeting. Biomaterials. 2017; 130:67-75.
Vasan N, Baselga J, Hyman D M. A view on drug resistance in cancer. Nature. 2019; 575(7782):299-309.
Graff C P, Wittrup K D. Theoretical analysis of antibody targeting of tumor spheroids: Importance of dosage for penetration and affinity for retention. Cancer Research. 2003; 63:1288-96.
Howe A, Sofou S. A nonintuitive combination of delivery-carriers for alpha-particle radiotherapy against metastatic and/or recurrent trastuzumab-resistant HER2-positive breast cancer. to be submitted. 2020.
Navarro-Teulon I, Lozza C, Pelegrin A, Vives E, Pouget J-P. General overview of radioimmunotherapy of solid tumors. Immunotherapy. 2013; 5(5):467-87.
Kratochwil C, Bruchertseifer F, Giesel F L, Weis M, Verburg F A, Mottaghy F, Kopka K, Apostolidis C, Haberkorn U, Morgenstern A. 225Ac-PSMA-617 for PSMA targeting alpha-radiation therapy of patients with metastatic castration-resistant prostate cancer. Journal of Nuclear Medicine. 2016; 57:1941-4.
Poty S, Francesconi L C, McDevitt M R, Morris M J, Lewis J S. α-Emitters for Radiotherapy: From Basic Radiochemistry to Clinical Studies-Part 2. J Nucl Med. 2018; 59(7):1020-7.
McDevitt M R, Sgouros G, Sofou S. Targeted and Nontargeted α-Particle Therapies. Annual Review of Biomedical Engineering. 2018; 20(1):73-93.
Song H, Hedayati M, Hobbs R F, Shao C, Bruchertseifer F, Morgenstern A, DeWeese T L, Sgouros G. Targeting Aberrant DNA Double-Strand Break Repair in Triple-Negative Breast Cancer with Alpha-Particle Emitter Radiolabeled Anti-EGFR Antibody. Molecular Cancer Therapeutics. 2013; 12(10):2043-54.
Fournier C, Zahnreich S, Kraft D, Friedrich T, Voss K O, Durante M, Ritter S. The Fate of a Normal Human Cell Traversed by a Single Charged Particle. Scientific Reports. 2012; 2:643.
Humm J L. A microdosimetric model of astatine-211 labeled antibodies for radioimmunotherapy. Int J Radiat Oncol Biol Phys. 1987; 13:1767-73.
Humm J L, Chin L M. A model of cell inactivation by alpha-particle internal emitters. Radiat Res. 1993; 134:143-50.
Macklis R M, Kinsey B M, Kassis Al, Ferrara J L, Archer R W, Hines J J, Coleman C N, Adelstein S J, Burakoff S J. Radioimmunotherapy with alpha-particle-emitting immunoconjugates. Science. 1988; 240:1024-6.
Sofou S. Radionuclide carriers for targeting of cancer. Int J Nanomed. 2008; 3:181-99.
Sgouros G. α-Particle-Emitter Radiopharmaceutical Therapy: Resistance Is Futile. Cancer Research. 2019; 79(21):5479-81.
Yard B D, Gopal P, Bannik K, Siemeister G, Hagemann U B, Abazeed M E. Cellular and Genetic Determinants of the Sensitivity of Cancer to α-Particle Irradiation. Cancer Research. 2019; 79(21):5640-51.
Poty S, Francesconi L C, McDevitt M R, Morris M J, Lewis J S. α-Emitters for Radiotherapy: From Basic Radiochemistry to Clinical Studies-Part 2. Journal of Nuclear Medicine. 2018; 59(7):1020-7.
Bandekar A, Karve S, M.-Y. C, Mu Q, Rotolo J, Sofou S. Antitumor efficacy following the intracellular and interstitial release of liposomal doxorubicin. Biomaterials. 2012; 33:4345-52.
Karve S, Alaouie A, Zhou Y, Rotolo J, Sofou S. The use of pH-triggered leaky heterogeneities on rigid lipid bilayers to improve intracellular trafficking and therapeutic potential of targeted liposomal immunochemotherapy. Biomaterials. 2009; 30:6055-64.
Karve S, Bajagur Kempegowda G, Sofou S. Heterogeneous domains and membrane permeability in phosphatidylcholine-phosphatidic acid rigid vesicles as a function of pH and lipid chain mismatch Langmuir. 2008; 24:5679-88.
Karve S, Bandekar A, Ali M R, Sofou S. The pH-dependent association with cancer cells of tunable functionalized lipid vesicles with encapsulated doxorubicin for high cell-kill selectivity. Biomaterials. 2010; 31:4409-16.
Bandekar A, Sofou S. Floret-shaped solid domains on giant fluid lipid vesicles induced by pH. Langmuir. 2012; 28:4113-22.
Stras S, Holeran T, Howe A, Sofou S. Interstitial Release of Cisplatin from Triggerable Liposomes Enhances Efficacy Against Triple Negative Breast Cancer Solid Tumor Analogues. Molecular Pharmaceutics. 2016; 13:3224-33.
Helmlinger G, Yuan F, Dellian M, Jain R K. Interstitial pH and pO2 gradients in solid tumors in vivo: high-resolution measurements reveal a lack of correlation. Nature medicine. 1997; 3:177-82.
Vaupel P, Kallinowski F, Okunieff P. Blood Flow, Oxygen and Nutrient Supply, and Metabolic Microenvironment of Human Tumors: A Review. Cancer Res. 1989; 49(23):6449-65.
Bailey A L, Cullis P R. Modulation of Membrane Fusion by Asymmetric Transbilayer Distributions of Amino Lipids. Biochemistry. 1994; 33(42):12573-80.
Bailey A L, Cullis P R. Membrane fusion with cationic liposomes: Effects of target membrane lipid composition. Biochemistry. 1997; 36:1628-34.
Sokolova V, Kozlova D, Knuschke T, Buer J, Westendorf A M, Epple M. Mechanism of the uptake of cationic and anionic calcium phosphate nanoparticles by cells. Acta Biomaterialia. 2013; 9(7):7527-35.
Lin J, Alexander-Katz A. Cell Membranes Open “Doors” for Cationic Nanoparticles/Biomolecules: Insights into Uptake Kinetics. ACS Nano. 2013; 7(12):10799-808.
Prabhakar U, Blakey D C, Maeda H, Jain R K, Sevick-Muraca E M, Zamboni W, Farokhzad O C, Barry S T, Gabizon A, Grodzinski P. Challenges and key considerations of the enhanced permeability and retention effect (EPR) for nanomedicine drug delivery in oncology. Cancer Research. 2013; 73:2412-7.
Lee H, Shields A F, Siegel B A, Miller K D, Krop I, Ma C X, LoRusso P M, Munster P N, Campbell K, Gaddy D F, Leonard S C, Geretti E, Blocker S J, Kirpotin D B, Moyo V, Wickham T J, Hendriks B S. 64Cu-MM-302 Positron Emission Tomography Quantifies Variability of Enhanced Permeability and Retention of Nanoparticles in Relation to Treatment Response in Patients with Metastatic Breast Cancer. Clinical Cancer Research. 2017; 23:4190-4202.
Basu S, Chen W, Tchou J, Mavi A, Cermik T, Czerniecki B, Schnall M, Alavi A. Comparison of triple-negative and estrogen receptor-positive/progesterone receptor-positive/HER2-negative breast carcinoma using quantitative fluorine-18 fluorodeoxyglucose/positron emission tomography imaging parameters. Cancer. 2008; 112(5):995-1000.
Cutruzzolà F, Giardina G, Marani M, Macone A, Paiardini A, Rinaldo S, Paone A. Glucose Metabolism in the Progression of Prostate Cancer. Frontiers in Physiology. 2017; 8:97.
Ippolito J E, Brandenburg M W, Ge X, Crowley J R, Kirmess K M, Som A, D'Avignon D A, Arbeit J M, Achilefu S, Yarasheski K E, Milbrandt J. Extracellular pH Modulates Neuroendocrine Prostate Cancer Cell Metabolism and Susceptibility to the Mitochondrial Inhibitor Niclosamide. PLOS ONE. 2016; 11(7):e0159675.
Estrella V, Chen T, Lloyd M, Wojtkowiak J, Cornnell H H, Ibrahim-Hashim A, Bailey K, Balagurunathan Y, Rothberg J M, Sloane B F, Johnson J, Gatenby R A, Gillies R J. Acidity Generated by the Tumor Microenvironment Drives Local Invasion. Cancer Research. 2013; 73(5):1524-35.
Vaupel P. Tumor microenvironmental physiology and its implications for radiation oncology. Semin Radiat Oncol. 2004; 14:198-206.
Jaggi J, Kappel B J, McDevitt M R, Sgouros G, Flombaum C D, Cabassa C, Scheinberg D A. Efforts to control the errant products of a targeted in vivo generator. Cancer Res. 2005; 65:4888-95.
Back, T. and L. Jacobsson, J. Nucl Med., 51(10): 1616-1623 (2010).
Wang R, Coderre J A. A Bystander Effect in Alpha-Particle Irradiations of Human Prostate Tumor Cells. Radiation Research. 2005; 164:711-722.
Thurber G M, Zajic S C, Wittrup K D. Theoretic Criteria for Antibody Penetration into Solid Tumors and Micrometastases. J Nucl Med. 2007; 48:995-999.
Borchardt P, Yuan R, Miederer M, McDevitt M, Scheinberg D. Targeted actinium-225 in vivo generators for therapy of ovarian cancer. Cancer Res. 2003; 63:5084.
Behling K, Maguire W F, López Puebla J C, et al. Vascular Targeted Radioimmunotherapy for the Treatment of Glioblastoma. Journal of Nuclear Medicine. 2016; 57:1576-1582.
Josefsson A, Nedrow J, Ray S, et al. Pharmacokinetic variability 225Ac and 213Bi from vector labeled 225Ac cancer therapy as a function of vector type (antibody vs. small molecule). Journal of Nuclear Medicine. 2018; 59:137-137.
Schwartz J, Jaggi J S, O'Donoghue J A, et al. Renal uptake of Bismuth-213 and its contribution to kidney radiation dose following administration of actinium-225-labeled antibody. Physics in Medicine and Biology. 2011; 56:721-733.
Jurcic J G. Targeted Alpha-Particle Therapy for Hematologic Malignancies. Seminars in Nuclear Medicine. 2020; 50:152-161.
Kratochwil C, Haberkorn U, Giesel F L. 225Ac-PSMA-617 for Therapy of Prostate Cancer. Seminars in Nuclear Medicine. 2020; 50:133-140.
Zhu C, Bandekar A, Sempkowski M, Ray Banerjee S, Pomper M G, Bruchertseifer F, Morgenstern A, Sofou S, Nanoconjugation of PSMA-targeting ligands enhances perinuclear localization and improves efficacy of delivered alpha-particle emitters against tumor endothelial analogues, Molecular Cancer Therapeutics 15 (2016) 106-113.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for inhibiting cancer cell growth, the method comprising contacting one or more cancer cells with a therapeutically effective amount of a first composition comprising a nanoparticle encapsulating an anti-cancer agent and a second composition comprising an antibody that binds to a cancer-specific receptor and is conjugated to the same anti-cancer agent comprising the first composition, whereby the first and second compositions are delivered to the cancer cells, thereby inhibiting cancer cell growth.

2. The method of claim 1, wherein the anti-cancer agent comprises a radiopharmaceutical agent.

3. The method of claim 2, wherein the anti-cancer agent comprises an alpha-particle emitting radiopharmaceutical agent.

4. The method of claim 3, wherein the alpha-particle emitting radiopharmaceutical agent comprises Actinium-225 (²²⁵Ac).

5. The method of claim 1, wherein the anti-cancer agent comprises a chemotherapeutic agent.

6. The method of any one of claims 1-5, wherein the nanoparticle comprises a cationic polymer attached to the surface thereof.

7. The method of claim 6, wherein the cationic polymer comprises polyethylene glycol (PEG) conjugated to dimethyl ammonium propane (DAP).

8. The method of any one of claims 1-7, wherein the nanoparticle comprises a pH-responsive membrane capable of forming phase-separated domains upon pH lowering.

9. The method of any one of claims 1-9, wherein the nanoparticle adheres to extracellular matrix of the one or more cancer cells.

10. The method of any one of claims 1-9, wherein the anti-cancer agent is released from the nanoparticle into the interstitium of the cancer cells.

11. The method of any one of claims 1-10, wherein the antibody binds to a cancer-specific receptor selected from HER2, epidermal growth factor receptor (EGFR), vascular endothelial growth factor receptor (VEGFR), interleukin-4 (IL-4), αvβ3 integrin, insulin-like growth factor receptor 1 (IGFR1), insulin-like growth factor receptor 2 (IGFR1), folate receptor, transferrin receptor, estrogen receptor, CXCR4, interleukin-6 (IL-6), transforming growth factor-beta receptor (TGF-βR), prostate specific membrane antigen (PSMA), α6β1 integrin, IGF1, EphA2, tumor necrosis factor-related apoptosis-inducing ligand (TRAIL), platelet derived growth factor receptor (PDGFR), CD20, and fibroblast growth factor receptor (FGFR).

12. The method of any one of claims 1-11, wherein the antibody is trastuzumab, cetuximab, panitumumab, rituximab, or bevacizumab.

13. The method of any one of claims 1-12, wherein the one or more cancer cells are from a primary cancer or tumor.

14. The method of claim 13, wherein the primary cancer or tumor is located in the breast, pancreas, or prostate.

15. The method of any one of claims 1-12, wherein the one or more cancer cells are from a metastatic cancer or tumor.

16. The method of any one of claims 1-15, wherein the one or more cancer cells are contacted with the anti-cancer agent in vitro.

17. The method of any one of claims 1-15, wherein the one or more cancer cells are contacted with the anti-cancer agent in vivo.

18. The method of claim 17, wherein the one or more cancer cells are in a human.

19. The method of any one of claims 1-18, wherein delivery of the first composition and the second composition to the one or more cancer cells synergistically lowers the therapeutically effective amount of the anti-cancer agent relative to a therapeutically effective amount of the anti-cancer agent administered in either the first composition or the second composition alone.

20. The method of any one of claims 1-19, wherein the first composition and the second composition are contacted with the one or more cancer cells simultaneously.

21. The method of any one of claims 1-19, wherein the first composition and the second composition are contacted with the one or more cancer cells sequentially.
